(19) United States
(12) Patent Application Publication
(10) Pub. No.: US 2025/0377295 A1
(43) Pub. Date: Dec. 11, 2025

(54) SYSTEM AND METHOD FOR CANNABIS CLASSIFICATION

(71) Applicant: THE STATE OF ISRAEL, MINISTRY OF AGRICULTURE & RURAL DEVELOPMENT, AGRICULTURAL RESEARCH ORGANIZATI, Rishon Lezion (IL)

(72) Inventors: Jakob SHIMSHONI, Rishon Letzion (IL); Tarin PAZ KAGAN, Meitar (IL); Matan BIRENBOIM, Ramat Gan (IL); David KENGISBUCH, Gimzo (IL); Yaira CHEN, Rosh Haayn (IL); Alona SADEH, Holon (IL); Dalia MAURER, Holon (IL); Daniel CHALUPOWICZ, Modiin (IL)

(21) Appl. No.: 18/878,279

(22) PCT Filed: Jun. 28, 2023
(86) PCT No.: PCT/IL2023/050666

§ 371 Date: Dec. 23, 2024

Related U.S. Application Data
(60) Provisional application No. 63/367,257, filed on Jun. 29, 2022.

(51) Int. Cl. G01N 21/3563; G01N 1/28; G01N 21/35; G01N 21/84; G01N 33/00

(52) U.S. Cl. CPC G01N 21/3563; G01N 1/286; G01N 21/84; G01N 33/0098; G01N 2001/2866; G01N 2021/3595; G01N 2021/8466; G01N 2201/1296

(57) ABSTRACT

A method and respective system are described. The method provides classification of cannabis inflorescence, and comprising: grinding said cannabis inflorescence; and determining a spectrogram of ground cannabis inflorescence; and providing data indicative of said spectrogram to trained machine learning system, pretrained on classification of material composition of cannabis inflorescence, to thereby obtain output data indicative of at least one of composition of selected cannabinoids and terpenes in said cannabis inflorescence, and varieties of said cannabis inflorescence.

### TECHNOLOGICAL FIELD

The present disclosure relates to systems and methods for the classification of cannabis inflorescence and cultivars and specifically relates to the classification of cannabis cultivars and their chemical composition of active compounds using spectroscopic analysis of cannabis inflorescence.

### BACKGROUND

Cannabis is an annual, dioecious, flowering herb in the family Cannabaceae. According to scientific consensus, Cannabis consists only of a single species, Cannabis sativa L., which has been botanically subdivided into three subspecies: Cannabis sativa, Cannabis indica, and Cannabis ruderalis. Commercially available medicinal cannabis cultivars are hybrids of sativa and indica ancestors and, therefore, the distinction between sativa and indica is no longer botanically valid. Today, more than 700 cultivated varieties (cultivars) of cannabis have been cataloged, each with potentially different effects.

The medical use of cannabis-based products has become widely accepted in recent years. Many commercial cannabis cultivars have been described in the literature and are currently used for recreational and medicinal purposes worldwide. Despite the enormous variety of cannabis-based products available (i.e., tinctures, oil, extracts, tablets, dried inflorescence), dried cannabis inflorescence is still the dominant form used for medical applications. This is primarily due to patient preference and also reflects the fact that the entire inflorescence provides greater therapeutic benefits than isolated phytocannabinoids, due to the presence of co-occurring bio-active plant substances such as terpenes. The therapeutic potential of medicinal cannabis has been demonstrated for treating of various medical conditions such as sleep disorders, nausea, anorexia, emesis, pain, inflammation, neurodegenerative disorders, epilepsy, and cancer. Cannabis inflorescences are rich in secondary metabolites representing a variety of classes of compounds, such as cannabinoids (>120), terpenes/terpenoids (>120), flavonoids (˜34), and poly-phenolic compounds (˜42).

The major cannabinoids (−)-Δ9-trans-tetrahydrocannabinol (THC), cannabidiol (CBD), cannabigerol (CBG), and cannabichromene (CBC) and their corresponding acidic compounds (i.e., THCA, CBDA, CBCA, and CBGA) are thought to be responsible for the main pharmacological properties of cannabis products. They act in conjunction with co-occurring terpenes and minor cannabinoids. Terpenes are highly volatile compounds responsible for the typical smell and taste of cannabis. Terpenes have a wide range of biological functions in plants, including roles in growth modulation, defense against herbivory, disease resistance, the attraction of pollinators, and, potentially, plant-plant communication and antioxidant properties. In humans and animals, terpenes are suspected to modulate the effects of other cannabinoids such as THC and CBD, and a phenomenon referred to as entourage effects. The current classification of medicinal cannabis cultivars is based on measured concentrations of total THC (i.e., the sum of THCA and THC normalized to their corresponding molecular weight) and total CBD (i.e., the sum of CBDA and CBD normalized to their molecular weight) and their corresponding ratio. Based on the ratio of THC to CBD, cultivars are classified into three internationally and nationally recognized classes: high THC, high CBD, and hybrid. Recently, a fourth primary therapeutic cannabis class has been made commercially available. That class is characterized by CBG concentrations that are more than 10-fold greater than the concentrations of other cannabinoids, as well as total THC and CBD levels below 1%.

At present, the elucidation of the chemical composition of medicinal cannabis is achieved by laborious, expensive, and time-consuming technologies, such as high-pressure liquid-chromatography-PDA (HPLC-PDA) and gas chromatography-mass spectroscopy (GC-MS). These methods also involve using hazardous solvents, such as acetonitrile methanol and possibly hexane, to achieve optimal analytical performance. The costs associated with the acquisition, maintenance, and operation of the instruments mentioned above are enormous. In addition, highly trained personnel are required for the daily operation of those instruments.

Various techniques were developed for characterizing the content of major cannabinoids in cannabis samples. These techniques generally avoid characterization of terpenes content of the samples, and generally require high time and labor.

### GENERAL DESCRIPTION

The Fourier transform near-infrared spectroscopy (FT-NIR) method uses the near-infrared (i.e., NIR; 700-1100 nm) and short-wave infrared (i.e., SWIR; 1100-2500 nm) regions of the electromagnetic spectrum. FT-NIR is widely applied to analyze samples containing organic compounds possessing a wide range of functional groups (aromatic, CIO, CIC, CH, NAH, NO, SUH, and OH), to determine quality parameters, as well as the content levels of specific compounds of interest. FT-NIR has several major advantages over chromatographic methods, such as minimal sample preparation that requires only homogenized dried samples (powders) or raw liquid samples (milk, alcoholic beverages, honey, etc.), which allows for rapid spectrum acquisition and data analysis (e.g., less than a minute). Furthermore, the operation and data analysis can be easily conducted following a simple procedure. However, to achieve highly accurate classification of a cannabis inflorescence classification and an accurate assessment of the concentrations of compounds of interest, a prior multivariate statistical and machine-learning approach is needed to handle the complexity of the data.

FT-NIR is used in chemometrics to construct classification and regression models, to predict target attributes. The classification models are used to group spectral signatures into categories, and regression models are used to model the spectral signature of a target based on specific chemical properties. These procedures involve the measured concentrations determined by chromatographic analytical methods, and their corresponding NIR spectra must be examined to develop reliable prediction models. Therefore, to characterize an unknown sample by near-infrared spectroscopy (NIRS) and obtain its spectrum, it is necessary to use a statistical model based on a large dataset (>300 samples) constructed to predict the sample properties. Chemometric-based multivariate classification and regression models such as partial least square-discriminant analysis (PLS-DA) and partial least square regression (PLS-R) are the most common and widely accepted approaches for predicting the properties of samples based on their NIRS spectra.

In recent years, numerous studies regarding the development of models for the prediction of cannabinoids using FT-NIR coupled with PLS-DA or PLS-R have been reported. However, those studies were conducted using small datasets (<200), focused on THC and CBD content, and did not allow for the separate prediction of acidic and neutral forms. Several of the aforementioned studies reported poor predictions of the cannabinoid concentrations in cannabis inflorescences. Moreover, the prediction of terpene contents has been completely neglected and has not previously been evaluated using FT-NIR.

In light of these knowledge gaps, the objective of the present study was to develop a straightforward, accurate, fast, and relatively cheap technique for the classification of cannabis cultivars and the prediction of a wide range of 10 cannabinoids and 9 terpenes utilizing FT-NIR technology combined with chemometrics and a relatively large dataset (325 samples). If this method is successful, FT-NIR could eventually replace laborious and expensive analytical tools for quality control of medicinal cannabis inflorescences, similar to how this technology is widely used for other pharmaceutical applications and in the food industry.

Accordingly, the present disclosure provides a system and corresponding method suitable for characterizing the active contents of cannabis inflorescence. The present disclosure utilizes Fourier Transform Infrared (FT-IR) spectroscopy and processing used for training machine learning modules allowing high-resolution classification of both major cannabinoids and terpenes. According to the present disclosure, the characterization technique enables the classification of inflorescence to chemovars of cannabis plants.

Accordingly, the present disclosure provides a method and respective system, for use in classification of cannabis inflorescence, the method comprises grinding a dried sample of cannabis inflorescence, e.g., containing up to 25% moisture or up to 22% moisture, generally under cryogenic/freezing conditions after brief immersion of the inflorescence in liquid nitrogen. The ground inflorescence is inspected by infrared spectroscopy to determine a respective spectrogram. The spectrogram of the cannabis inflorescence has indications of various functional groups such as aromatic, C\\O, C\\C, C\\H, N\\H, N\\O, S\\H, and OH groups of materials present in the sample. The spectrogram is then processed using suitably trained one or more machine learning modules to provide output data on a plurality of cannabinoids and terpenes in the sample.

One of the major advantages of classification cannabis inflorescence based on FT-NIR spectroscopy as described herein, is that the sample preparation required is simplified over the conventional techniques. According to some embodiments of the present disclosure, sample preparation requires only homogenous grinding of the dried frozen cannabis inflorescence. This differs from conventional techniques such as chromatographic determinations, which require extensive extraction and cleaning procedures. Hence, according to the present disclosure, the technique provides an alternative to the laborious conventional wet chromatographic analysis currently used to assess Cannabis sativa L. classes/chemovars and chemical composition. The present technique can provide a rapid chemical-composition analysis tool for both consumers and farmers, assisting with breeding processes and kinetic studies for evaluating cannabinoid and terpene concentrations in real-time.

Thus, according to a broad aspect, the present disclosure provides a method for use in the classification of cannabis inflorescence, the method comprises:
grinding said cannabis inflorescence;
determining a spectrogram of ground cannabis inflorescence;
providing data indicative of said spectrogram to trained machine learning system, pretrained on classification of material composition of cannabis inflorescence, to thereby obtain output data indicative of at least one of composition of selected cannabinoids and terpenes in said cannabis inflorescence, and varieties of said cannabis inflorescence.

According to some embodiments, grinding said cannabis inflorescence comprises grinding said cannabis inflorescence after freezing in liquid nitrogen.

According to some embodiments, grinding said cannabis inflorescence comprises grinding to a predetermine powder size in the range of 1-10 micrometer.

According to some embodiments, said determining a spectrogram of ground cannabis inflorescence comprises obtaining a Fourier Transform Infrared spectroscopic (FT-NIR) data of said ground cannabis inflorescence.

According to some embodiments, said determining a spectrogram of ground cannabis inflorescence comprises obtaining an absorption said spectrogram using monochromator spectrometer.

According to some embodiments, said spectrogram comprises wavelength range between 1000 nm to 2500 nm.

According to some embodiments, the method may further comprise preprocessing of said spectrogram, said processing comprises at least one of signal amplification and thresholding of the spectrogram data.

According to some embodiments, said preprocessing further comprises applying smoothing operation on at least one of said spectrogram, first derivative and second derivative thereof.

According to some embodiments, said trained machine learning system may be trained on a labeled data set comprising a plurality of cannabis inflorescence of a plurality of cannabis cultivar/varieties labeled by respective chemovar of said plurality of cannabis inflorescence.

According to some embodiments, the respective chemovar may be determined by at least one mass spectrometry and chromatography measurement of said plurality of cannabis inflorescence.

According to some embodiments, said trained machine learning system may comprise a plurality of processing routes, each processing route being directed for quantifying a selected one of cannabinoids and terpenes in said cannabis inflorescence.

According to some embodiments, said preprocessing may comprise generating a plurality of cropped copies of said data indicative of said spectrogram, wherein each of said cropped copies is cropped around one or more characteristic wavelength ranges indicative of absorption of a respective one of said selected cannabinoids and terpenes in said cannabis inflorescence.

According to one other broad aspect, the present disclosure provides a system for classification of cannabis inflorescence, comprising at least one processor, a memory unit, associated with and one or more input/output connections, wherein said at least one processor is configured and operable for receiving input data indicative of one or more spectrograms taken from one or more cannabis inflorescence samples, and processing said input data to determine quantitative data on one or more cannabinoid and terpene composition of said one or more cannabis inflorescence; wherein said processing comprises utilizing at least one pre-trained machine learning module pretrained on the classification of a material composition of cannabis inflorescence.

According to some embodiments, said processing further comprises preprocessing of input spectrogram, said preprocessing comprises at least one of signal amplification and thresholding of said one or more spectrograms.

According to some embodiments, said preprocessing further comprises applying smoothing operation on said one or more spectrograms, first derivative and second derivative thereof.

According to some embodiments, said at least one pre-trained machine learning module comprises a plurality of processing routes, each processing route being directed for quantifying a selected one of cannabinoids and terpenes in said cannabis inflorescence.

According to some embodiments, said at least one processor is configured and operable for preprocessing said one or more spectrograms and for generating a plurality of cropped copies of said one or more spectrograms, wherein each of said cropped copies is cropped around one or more characteristic wavelength ranges indicative of absorption of a respective one of said selected cannabinoids and terpenes in said cannabis inflorescence.

According to some embodiments, said at least one processor is configured and operable for one or more spectrograms and for generating a plurality of cropped copies of said data indicative of said spectrogram, wherein each of said cropped copies is cropped around one or more characteristic wavelength ranges indicative of absorption of a respective one of said selected cannabinoids and terpenes in said cannabis inflorescence.

According to some embodiments, the system may further comprise an infrared spectrometer unit connectable to said at least one processor via one or more communication lines; said infrared spectrometer unit comprises a sample mount for holding a sample and is configured to selective measure sample absorption in a selected wavelength range within infrared spectrum thereby generating spectrogram data indicative of one or more spectrograms taken from one or more cannabis inflorescence samples and transmitting said spectrogram data to said at least one processor.

According to some embodiments, said infrared spectrometer unit is a Fourier Transform Infrared spectrometer unit.

According to yet another broad aspect, the present invention provides a computer implemented method for use in classification of cannabis inflorescence, comprising:
receiving input data indicative of one or more infrared spectrograms of cannabis inflorescence;
processing said input data to determine at least one of composition of selected cannabinoids and terpenes in said cannabis inflorescence, and cultivar of said cannabis inflorescence; and
generating output data indicative of said at least one of composition of selected cannabinoids and terpenes in said cannabis inflorescence, and varieties of said cannabis inflorescence;
wherein, said processing comprises operating at least one machine learning module, pretrained for classification of material composition of cannabis inflorescence, to determine quantitative data on selected number of cannabinoids and terpenes in said cannabis inflorescence.

According to some embodiments, the at least one machine learning module comprises a plurality of processing routes, each processing route being directed for quantifying a selected one of cannabinoids and terpenes in said cannabis inflorescence.

According to some embodiments, said processing comprises at least one preprocessing stage, comprising generating a plurality of cropped copies of said one or more infrared spectrograms, wherein each of said cropped copies is cropped around one or more characteristic wavelength ranges indicative of absorption of a respective one of said selected cannabinoids and terpenes in said cannabis inflorescence.

According to some embodiments, said processing comprises at least one preprocessing stage, comprising applying smoothing operation on at least one of said spectrogram, first derivative and second derivative thereof.

According to a further broad aspect, the present disclosure provides a program storage device readable by machine, tangibly embodying a program of instructions executable by one or more computer processors, comprising:
receiving input data indicative of one or more infrared spectrograms of cannabis inflorescence;
processing said input data to determine at least one composition of selected cannabinoids and terpenes in said cannabis inflorescence, and cultivar of said cannabis inflorescence; and
generating output data indicative of said at least one composition of selected cannabinoids and terpenes in said cannabis inflorescence, and varieties of said cannabis inflorescence;
wherein, said processing comprises operating at least one machine learning module, pretrained for classification of material composition of cannabis inflorescence, to determine quantitative data on selected number of cannabinoids and terpenes in said cannabis inflorescence.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 schematically illustrates a system for classifying cannabis inflorescence according to some embodiments of the present disclosure;

FIG. 2 illustrates a method for classifying cannabis inflorescence according to some embodiments of the present disclosure;

FIG. 3 Exemplifies a process for training a machine learning system for classifying cannabis inflorescence according to some embodiments of the present disclosure schematically;

FIG. 4 exemplifies a Fourier transform infrared spectroscopic system for use in classifying cannabis according to some embodiments of the present disclosure;

FIGS. 5A to 5C show measured spectrograms (FIG. 5A), normalized spectrogram (FIG. 5B), and spectrogram following thresholding preprocessing (FIG. 5C) according to some embodiments of the present disclosure;

FIG. 6 shows mean cannabinoids concentrations measured by HPTC-PDA for different cannabis cultivars;

FIG. 7 shows mean terpene concentrations measured by HPTC-PDA for the cannabis cultivars;

FIG. 8 shows cross-validation score plot of the first three latent variables (LVs) obtained by the PLS-DA classification of cannabis by major classes through NIR spectra;

FIGS. 9A to 9O show correlations between the measured concentrations of cannabinoids (by HPLC-PDA; x-axis) and the concentrations predicted by PLS-R (y-axis) for different cannabinoids including CBCA (FIG. 9A), CBGA (FIG. 9B), THCA using full-range model (FIG. 9C), THCA using high-range model (FIG. 9D), THCA using mid-range model (FIG. 9E), THCA using low-range model (FIG. 9F), CBDA using full-range model (FIG. 9G), CBDA using high-range model (FIG. 9H), CBDA using low-range model (FIG. 9I), THC (FIG. 9J), CBD (FIG. 9K), CBG (FIG. 9L), CBTA (FIG. 9M), THCA-C4 (FIG. 9N), and CBGMA (FIG. 9O);

FIGS. 10A to 10K show correlations between the measured concentrations of terpenes (by GC-MS; x-axis) and the concentrations predicted by PLS-R (y-axis) for different terpenes including D-Limonene (FIG. 10A), Linalool (FIG. 10B), β-Caryophyllene (FIG. 10C), β-Pinene (FIG. 10D), α-Pinene using full-range model (FIG. 10E), α-Pinene using high-range model (FIG. 10F), α-Pinene using low-range model (FIG. 10G), β-myrcene (FIG. 10H), α-Humulene (FIG. 10I), Bisabolol (FIG. 10J), and Guaiol (FIG. 10K);

FIGS. 11A and 11B shows VIP spectral bands associated with absorption spectra of THC and THCA respectively;

FIG. 12 shows cross-validation score plots of the first two latent variables (LVs) obtained by the PLS-R models for different Cannabinoids; and

FIG. 13 shows cross-validation score plots of the first two latent variables (LVs) obtained by the PLS-R models for different terpenes.

### DETAILED DESCRIPTION OF EMBODIMENTS

As indicated above, the present disclosure provides systems and methods for use in classifying cannabis inflorescence. Reference is made to FIG. 1, illustrating schematically a system 100 according to some embodiments of the present disclosure. FIG. 1 illustrates a system 100, including a grinder unit 110, infrared spectrometer 115, and a processing unit 150. The grinder unit 110 may be a typical grinder, mortar, and pestle, ball grinder, or other grinding arrangements suitable for grinding cannabis inflorescence to provide a selected particle size. In some configurations, the grinder 110 may include an input port for accepting liquid air or liquid nitrogen for grinding the sample in generally cryogenic conditions. Following grinding of cannabis inflorescence to the desired size, typically in the range of 1-10 micrometers, the ground sample is inspected by infrared spectroscopy using infrared spectrometer 115. Generally, infrared spectrometer 115 includes at least a light source 120, the sample chamber 130, and detector 140. The spectrometer 115 may be configured as Fourier transform infrared spectrometer, replacing a wavelength selection arrangement, such as a prism or grating by an interferometer, thereby simplifying spectrometric sampling process. However, it should be understood that other types of infrared spectrometers may be used.

As illustrated, the detector 140 may be associated with a processing/computer unit 150, or be separated therefrom, and configured to generate output data indicative of the spectrogram of the tested sample. The spectrogram output data is transmitted to the processing unit 150 to determine quantitative data on one or more materials and material compositions in the tested sample. Processing unit 150 includes at least one processor and memory unit 180 operatively connected to a hardware-based I/O interface 190. Processing unit 150 is configured to provide processing necessary for operating the system 100 as further detailed herein and comprises one or more processors (not shown separately) and a memory. The one or more processors of processing unit 150 can be configured to execute several functional modules in accordance with computer-readable instructions implemented on a non-transitory computer-readable memory associated with or being part of the processing unit 150. Such functional modules are referred to hereinafter as comprised in the processing unit 150. The spectrometer 115 may generally provide spectrogram data, including data on sample absorption in near-infrared and short-wave infrared ranges. In some configurations, the spectrometer 115 is configured to provide spectrogram data, including data on absorption at a wavelength range between 1000-2500 nm, and in some embodiments, between 1000-2500 nm.

According to certain embodiments, the processing unit 150 may include at least a preprocessing module 160 and a machine learning module 170 configured for processing input spectrogram data to generate output data indicative of at least one of composition of selected cannabinoids and terpenes in said cannabis inflorescence, and varieties of said cannabis inflorescence. In this connection, the preprocessing module 160 may be configured to apply one or more selected preprocessing operations on the input spectrogram to generate modified spectrogram data. The machine learning module 170 is generally pre-trained on the classification of cannabis inflorescence to determine at least one composition of selected cannabinoids and terpenes in said cannabis inflorescence, as described in more detail below. The machine learning module 170 may thus generate output data indicative of the classification and material content of the inflorescence sample. The output data may be provided to an operator via that I/O port 190, stored in memory 180 and/or transmitted by network communication to one or more other systems for further processing.

In some configurations, the machine learning module 170 may be configured with a plurality of machine learning processing routes or a plurality of machine learning sub-modules, each trained for quantifying a selected one of a collection of cannabinoids and terpenes. In some further configurations, the machine learning module 170 may also include a classification and correlation module, trained for classifying the input data as relating to one of a selection of cannabis cultivars.

Further, in some configurations, the processing unit may utilize the preprocessing module 160 for preprocessing the input spectrogram data to transform the spectrogram data, thereby simplifying machine learning processing thereof. The preprocessing may include one or more preprocessing stages, associated with the configuration of the machine learning module and with one or more parameters of the input spectrogram data. Generally, the preprocessing may include at least one preprocessing action such as signal amplification and/or thresholding of the spectrogram data. More specifically, signal amplification and thresholding are directed at enhancing signal data associated with the absorption of impinging radiation by one or more functional groups of chemical existing in the inflorescence. Additionally, in case the input spectrogram data is noisy, the preprocessing may also include smoothing of the spectrogram curve or a first or second derivative of the spectrogram curve.

In some embodiments, the preprocessing may further include the selection of spectrogram sections containing VIP (Variable importance in projection). The selection is based on marking certain spectral sections of the spectrogram associated with identifying selected cannabinoids and/or terpenes. Accordingly, for each machine learning processing path, directed toward estimating the quantity of one or more cannabinoids or terpenes, selected spectral sections may be marked as VIP sections and given a score. A score greater than 1 (one) indicates high importance for the processing operation, while lower scores indicate that the spectral section is of low importance. For example, spectral regions between 1450-1880 nm and 2130-2350 nm are typically marked as VIP for estimation of cannabinoid compounds, and spectral sections between 1000-1210 nm are marked as VIP for estimation of terpenes compounds.

Additionally, or alternatively, in embodiments that utilize a plurality of machine learning processing routes, each processing route may utilize slightly different input data, to enhance processing accuracy for quantifying respective one of the cannabinoids and terpenes. Accordingly, in such configurations, the preprocessing module 160 may utilize a preprocessing stage associated with generating a plurality of copies of the input spectrogram, where each copy is cropped to mark data associated with selected one or more wavelength ranges indicative of the respective one of the one of the cannabinoids and terpenes.

Accordingly, the preprocessing may include applying one or more filters on the input spectrogram data, directed for removing information not relating to the material content of the sample, or emphasizing information pieces that relate specifically to selected materials, as well as linearizing the data and removing external sources of noise from the spectrogram data. For example, in a general configuration, in case the input spectrogram is noisy, the input spectrogram may be preprocessed for smoothing. Such smoothing preprocessing may be applied to the spectrogram itself or the first or second derivative thereof. Additionally, a typical input spectrogram may also be preprocessed to enhance absorption peaks associated with functional groups over background spectrogram data. Such preprocessing may utilize one or more selected peak detection techniques, such as autoscaling of spectrogram data to enhance peaks and thresholding the spectrogram by assigning data points with a value below a selected threshold with zero value maintaining values of data points above the selected threshold. In this connection, the preprocessing may utilize various algorithms for enhancing absorption peaks and reducing noise in the spectrogram data. For example, thresholding of the spectrogram data may utilize selected techniques such as Generalized Least Squares weighting (GLS-weighting). Generally, various other techniques may be used. However, preprocessing of the spectrogram data may be determined in accordance with corresponding preprocessing operations used for training a machine learning module for determining cannabinoids and terpenes content of cannabis inflorescence.

Generally, GLS-Weighting (GLSW) is a filter calculated based on the differences between samples that should otherwise be similar. These differences are considered interferences or “clutter” and the filter attempts to down weight (shrink) those interferences. A simplified version of GLSW is called External Parameter Orthogonalization (EPO), which does an orthogonalization (complete subtraction) of some number of significant patterns identified as clutter. A simplified version of EPO emulates the Extended Mixture Model (EMM), in which all identified clutter patterns are orthogonalized.

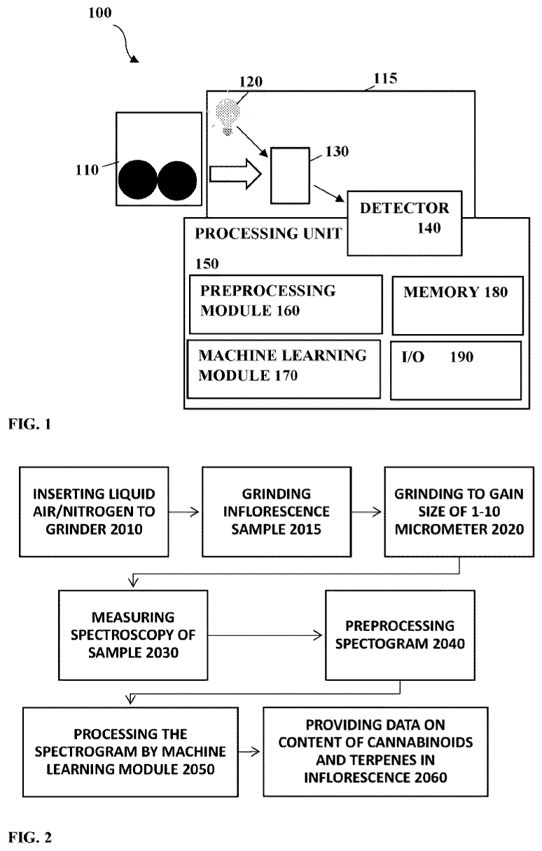

The method for quantifying a selected set of cannabinoids and terpenes in cannabis inflorescence is exemplified in FIG. 2 in the way of a block diagram. As illustrated, the present disclosure utilizes cannabis inflorescence and typically needs grinding the dried cannabis inflorescence 2015 to enable proper spectrometry thereof. Typically, the method may include adding liquid air or liquid nitrogen 2010 to the cannabis inflorescence to provide cryogenic grinding conditions. Further, according to the present technique, the grinding is performed to regain particle size of 1-10 micrometers 2020.

Generally, the cannabis inflorescence to be measured is dried for preservations and ease of use, by removing at least 77% of water content from the inflorescence prior to grinding. Using liquid air/nitrogen in grinding may simplify the grinding process and enable achieving uniform particle size. Additionally, grinding in cryogenic conditions freezes any humidity in the inflorescence, reducing interference associated with water absorption.

Proceeding with FIG. 2, the method further includes obtaining a spectrogram of the ground cannabis sample 2030. To this end, the cannabis sample may be placed within a spectrometer, typically operating in the visible to infrared wavelength range and determining absorption levels as a function of wavelength. The spectrometer may be any type of spectrometer operating in a selected wavelength range, typically including the range between 1000 nm and 2500 nm and preferably including the range between 1000 nm and 2500 nm. In chemical analysis, infrared spectroscopy enables the detection of a plurality of functional groups appearing in various chemical compounds. Following spectroscopic measurements, the method, according to some embodiments of the present disclosure, may utilize certain preprocessing of the spectrogram data 2040. The preprocessing is generally directed at removing noise that may be associated with the operation of the spectrometer used, as well as improving the signal-to-noise ratio with respect to absorption peaks of functional groups of other absorption sources and fluctuations in optical emission of the spectrometer. As indicated above, the preprocessing may include one or more processing operations directed at an increasing signal-to-noise ratio of the spectrogram data.

Accordingly, in some cases, depending on the operational parameters of the spectrometer used, the preprocessing may include applying a smoothing algorithm to the spectrogram or its first or second derivatives. The smoothing is directed to reduce noise associated with large variations in absorption between measurements and high-frequency variations in light source intensity.

Additional preprocessing operations may be used, including one or more selected normalization methods that enhance the spectrogram's features. Such normalization methods include, for example, mean centering and/or autoscaling. Following normalization, the preprocessing may further include thresholding or weighting processing directed at lowering the background data of the spectrogram with respect to absorption peaks associated with functional groups. Such weighting processing may include GLS-weighting or other weighting techniques.

Following preprocessing, the present disclosure utilized processing the spectrogram data using a pre-trained machine learning module 2050, for determining data on the presence and amount of one or more of a selected set of cannabinoids and terpenes for which the machine learning module is trained. The machine learning processing may utilize one or more machine learning topologies, including e.g., one or more neural networks or other machine learning topologies. The machine learning processing is typically configured to provide quantitative output data indicative of one or more cannabinoids and terpenes and may also provide output data indicative of the cultivar of the cannabis inflorescence sample 2060.

The machine learning processing may be associated with a plurality of processing routes. More specifically, every single route of the machine learning processing of the spectrogram data may be directed at quantifying one of a selected set of cannabinoids and terpenes or at classifying the cultivar of the sample.

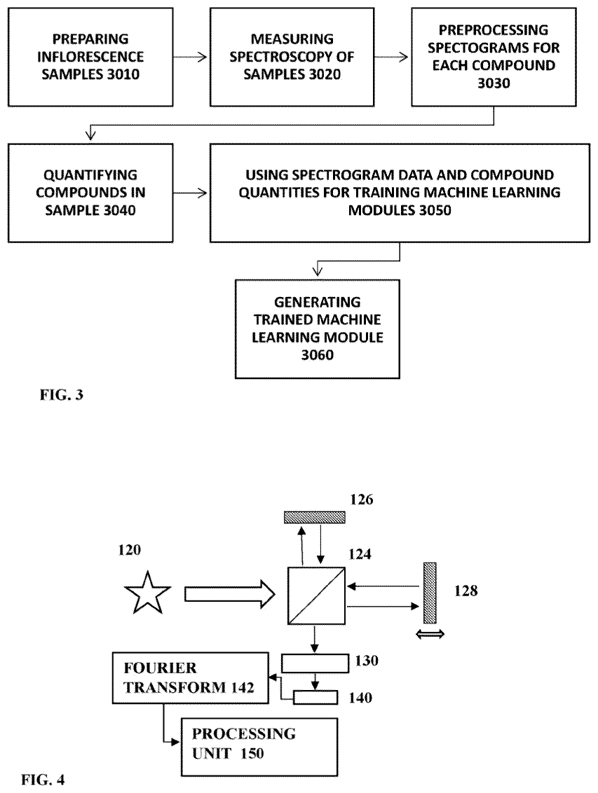

In this connection, each machine learning processing route may utilize a specifically trained machine learning module, trained for determining quantitative data of a selected chemical (typically cannabinoids or terpenes) based on spectrogram data. This is exemplified in FIG. 3 in the way of a block diagram. More specifically, a plurality of cannabis inflorescence, of different known cultivars are used for training the machine learning module. Each sample is prepared by grinding 3010, typically in the presence of liquid air/nitrogen, to the desired particle size of 1-10 micrometer. The samples are each measured by spectrometer 3020 to provide a respective plurality of spectrograms, and each spectrogram is preprocessed 3030 as described above.

In addition to the spectroscopy measurements, each of the plurality of samples is analyzed for the content of a selected set of cannabinoids and terpenes 3040. The analysis may be performed using chemometric techniques, e.g., mass spectrometry, chromatography, and any other technique. This analysis provides quantitative details of the selected compounds in each sample for use in training one or more machine learning modules. Generally, the spectrogram data pieces for the different samples are labeled by respective quantitative data on one or more selected compounds. The so-labeled data is used for training a machine learning module 3050. This enables the machine learning module to estimate the quantity of one or more of the compounds based on spectrogram input data. The training results in providing pretrained one or more machine learning module 3060 trained for processing spectrogram data of cannabis inflorescence and generate a quantitative estimate of selected set of cannabinoids and terpenes in respective cannabis inflorescence.

As indicated above, optical spectroscopic measurements generally obtain the spectrogram data on the cannabis inflorescence samples. FIG. 4 exemplifies a Fourier transform optical spectrometer being a part of a system for quantifying active compounds of cannabis inflorescence according to some embodiments of the present disclosure. As illustrated in FIG. 4, a light source unit 120 is positioned to direct broadband (while) illumination toward a beam splitting arrangement (beam splitter 124). The split light components follow two paths, the first path toward a fixed position mirror 126 and a second path toward a moving mirror 128 operated to move periodically within a selected range toward and away of the beam splitter 124. Reflected light merges at the beam splitter 124 again and is directed at sample 130. Light passing through the sample is absorbed, while a portion of the light is transmitted through the sample 130 and collected by a detector 140. The detector operates for collecting a plurality of sequential intensity data pieces along a period that covers at least one full period of the moving mirror 128. The collected intensity sequence is processed for determining a Fourier transform thereof 142, thereby providing a spectrogram of the sample. Determining the Fourier transform of collected intensity may be done digitally by processing the collected data or using suitable analog circuitry. The collected spectrogram is then transmitted to the processing unit 140 for processing and analysis as described herein to provide output data indicative of the quantity of selected chemical compounds including cannabinoids and terpenes, in the sample.

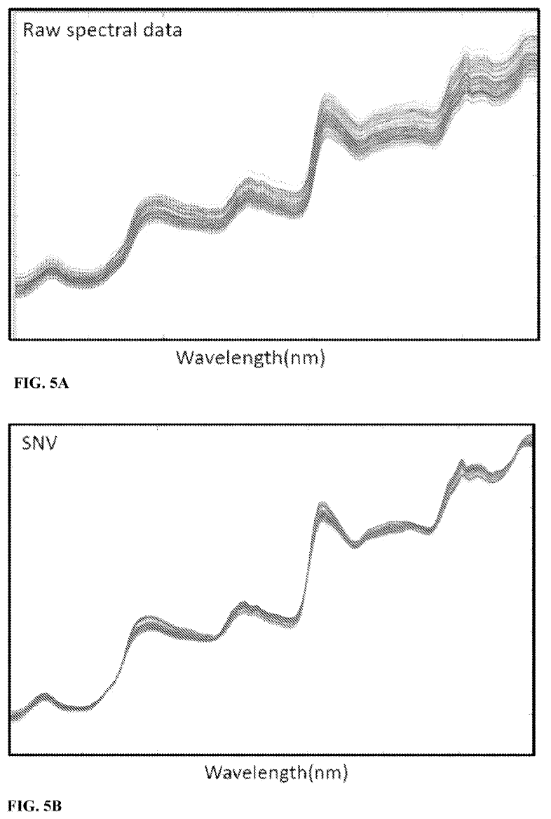

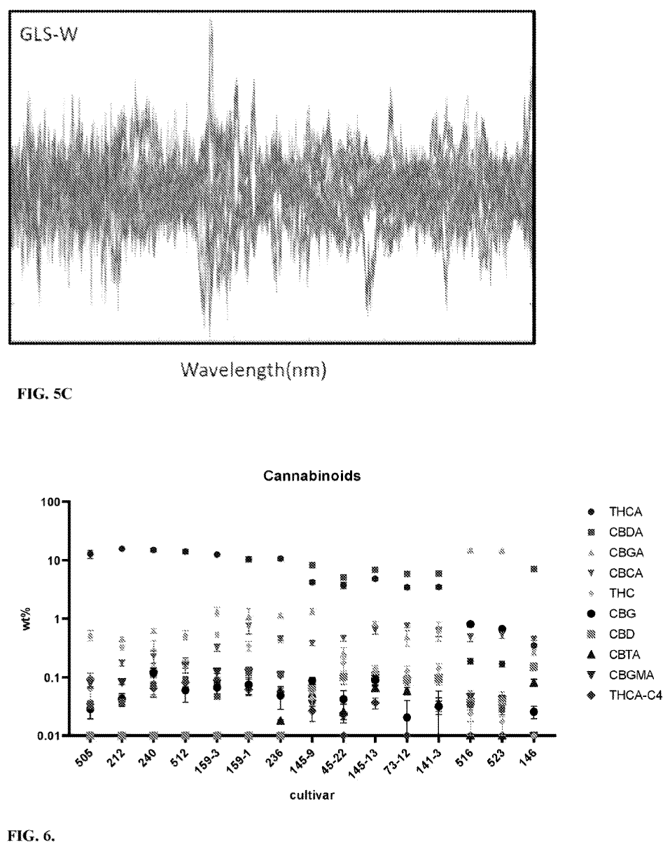

Reference is made to FIGS. 5A to 5C exemplifying raw spectrogram data measured by a FT-NIR spectrometer (FIG. 5A), preprocessed spectrograms, processed for normalization by standard normal variate (SNV) (FIG. 5B), and weighted spectrogram data, following preprocessing by GLS-Weighting (FIG. 5C). As shown, the variations in spectrogram between cultivars are very small, requiring proper processing for determining the chemical differences between them.

### Experimental

### Plant Material

Experimental study of the present disclosure is based on an inspection of commercial dried medicinal cannabis inflorescences of 15 different chemovars provided by the Bar-Lev farm (Kfar Hess, Israel). The cannabis inflorescences were all analyzed for their cannabinoid and terpene content at the Agricultural Research Organization Department of Food Safety (ARO, Volcani, Israel). The present experimental study was focused on commercially available cultivars, including high-THC (>15%) cultivars, hybrid-THC/CBD cultivars (˜5-9% total CBD and total THC), high-CBG cultivars (>15%), and high-CBD cultivars (>10%).

### Sample Preparation

The cannabis inflorescence samples (six inflorescences of each chemovar, weighing 3-6 g) were inserted into a mortar and liquid nitrogen was slowly added covering about a third of the mortar volume. After complete evaporation of the liquid nitrogen, the cannabis inflorescence was ground homogenously using a pestle in the. Generally grounding by ball grinder may also be used. From each of the 15 chemovars, 10-30 samples were prepared. For each sample, chemical compositions were analyzed, yielding a data set of 325 samples. The homogenous ground cannabis samples (100 mg±0.1) were inserted into a 2-mL glass vial and analyzed using a NIR spectrometer. For the determination of cannabinoid and terpene concentrations, the same homogenously ground cannabis samples (100±0.1 mg) used for the near-infrared spectroscopy (NIRS) spectra measurement were extracted with 4 mL of ethanol in 15-mL Falcon tubes and shaken (Digital Orbital Shaker, MRC, Israel) in the dark for 20 min at 500 rpm. The supernatant (1 mL) was transferred to an Eppendorf tube and centrifuged for 4 min at 13000 rpm. Subsequently, 0.25 mL of the supernatant was introduced into a GC vial for the terpene analysis and subjected to GC-MS analysis. For the cannabinoid analysis, the supernatant was diluted 1:5 with ethanol, and then 1 ml of the diluted supernatant was transferred to a HPLC vial and subjected to HPLC-PDA analysis.

### Instrumentation

The Fourier transform near-infrared (FT-NIR) spectral data were obtained using a ThermoFisher Antaris II FT-NIR Analyzer that is equipped with an integrated sphere and indium gallium-arsenic (In—Ga—As) detector. The reflectance spectra were measured with a resolution of 4 cm⁻¹ in the range of 10,000 cm⁻¹ to 4000 cm⁻¹ (or 1000-2500 nm). A total of 16 scans were performed for each measurement, and each sample was measured four times from different directions. The white reference background was obtained using a spectralon disc (a polystyrene disc) and measured between triplicate samples. Spectral absorbance values were recorded in reflectance mode as log 1/R, where R is the sample reflectance.

The ethanolic cannabis extracts were analyzed for cannabinoids using HPLC-PDA (Acquity Arc FTN-R; Model PDA-2998, Waters Corp., Milford, MA, USA) equipped with a Kinetex 1.7 μm XB-C18 100A LC column (150×2.1 mm i.d. and 1.7 μm particle size; Phenomenex, Torrance, CA, USA). The mobile phase consisted of formic acid, 20 mM ammonium formate buffer at pH 2.9 (mobile phase A), and acetonitrile (mobile phase B). The following isocratic program was applied: 30% A, 70% B, with a 16-min run time. The following parameters were used to quantify cannabinoids: a detection wavelength of 228 nm, a flow rate of 0.3 mL/min, and a 2-μL injection volume. The cannabinoid concentration in each sample was quantified by comparing the integrated peak area with the corresponding cannabinoid calibration curves ranging from 1 to 1000 mg/L (Table 1).

The terpene analysis was carried out by GC/MC (Agilent, Santa Clara, CA, USA). The GC/MS injector was operated at 250° C. under split-less conditions. The volatile analytes were separated on a DB-5 capillary column (5% phenyl, 95% dimethylpolysiloxane, 30 m×0.250 mm, 0.25 m; Agilent, Santa Clara, CA, USA) using the following temperature gradient. The gradient started at 50° C. for 1 min and increased at a rate of 1.5° C./min until 60° C., where it was held for 1 min, followed by a temperature increase at a rate of 3° C./min until 130° C., where it was held for 1 min. Subsequently, a temperature of 180° C. was attained at a rate of 2° C./min and held for 2 min.

The limit of detection (LOD) was estimated based on a 3:1 signal-to-noise ratio and the limit of quantification (LOQ) was calculated based on a 10:1 signal-to-noise ratio. Repeatability and accuracy were evaluated at four different concentrations: 5, 10, 50, and 100 mg/L. Each sample was analyzed five times within a single day, three times on three different days, and within-day and between-days; repeatability and accuracy were calculated. The quantification of detected cannabinoids and terpenes for which we lacked analytical-standard calibration curves was carried out using the calibration curves of compounds of similar structures and response trends reported in previously published studies.

TABLE 1
Analytical parameters of cannabinoids analyzed by UHPLC-PDA.

| | | | | | Concen- | | Repeat- | Repeat- |
|---|---|---|---|---|---|---|---|---|
| | | | | | tration | | ability | ability |
| | | | LOQ | LOD | range | | [% CV | [% CV |
| | RT | | [μg/mL | [μg/mL | [μg/mL | Accuracy | within | between |
| Name | [min] | R² | solvent] | solvent] | solvent] | [%] | day] | day] |
| CBTA | 2.09 | 0.999 | 1.21 | 0.36 | 1-1000 | 3% | 0.7% | 0.1% |
| CBDA | 3.6 | 0.999 | 1.21 | 0.36 | 1-1000 | 3% | 0.7% | 0.1% |
| CBGA | 3.9 | 0.999 | 1.55 | 0.46 | 1-100 | 2% | 0.6% | 0.2% |
| CBG | 4.1 | 0.999 | 2.57 | 0.77 | 1-100 | 2% | 0.5% | 0.2% |
| CBD | 4.4 | 0.999 | 2.68 | 0.80 | 1-500 | 10% | 0.6% | 0.08% |
| THCV | 4.5 | 0.999 | 2.51 | 0.63 | 1-100 | 11% | 0.7% | 0.1% |
| CBGMA | 5.5 | 0.999 | 1.21 | 0.36 | 1-1000 | 3% | 0.7% | 0.1% |
| CBN | 6.4 | 0.997 | 1.43 | 0.43 | 1-100 | 5% | 0.6% | 0.04% |
| THCA-C4 | 7.4 | 0.999 | 1.21 | 0.36 | 1-1000 | 3% | 0.7% | 0.1% |
| delta- | 8.2 | 0.996 | 3.69 | 1.11 | 1-500 | 8% | 1.4% | 1.9% |
| 9-THC | | | | | | | | |
| delta- | 8.5 | 0.999 | 3.12 | 1.05 | 1-100 | 10% | 0.8% | 0.2% |
| 8-THC | | | | | | | | |
| CBL | 9.8 | 0.996 | 2.60 | 0.81 | 1-100 | 10% | 0.7% | 0.1% |
| CBC | 10.4 | 0.996 | 2.54 | 0.85 | 1-100 | 7% | 0.4% | 0.2% |
| THCA | 10.7 | 0.999 | 2.52 | 0.76 | 1-1000 | 4% | 0.5% | 0.4% |
| CBCA | 12.4 | 0.996 | 9.50 | 3.15 | 5-100 | 5% | 0.8% | 0.2% |

RT is the retention time in minutes, LOQ is the limit of quantitation, and LOD is the limit of detection. Certain samples, including CBTA, CBGMA, and THCA-C4 were Quantified using CBDA.

5 The ion and injection source temperatures were 230° C. and 250° C., respectively. Helium was used as a carrier gas at a 1 mL/min flow rate. After verification with retention indices, the compounds were identified using NIST Atomic Spectra Database version 1.6 (U.S. Department of Commerce, Gaithersburg, MD, USA). The analyte concentration was determined by comparing the integrated peak area with the corresponding calibration curve ranging from 0.5 to 250 mg/L (Table 2). All terpenes presented accuracy values lower than 10% and within- and between-day repeatability lower than 1%.

TABLE 2
Analytical parameters of terpenes analyzed by GC/MS.

| | | | LOQ | LOD | Concentration |
|---|---|---|---|---|---|
| | RT | | [μg/mL | [μg/mL | range |
| Name | [min] | R² | solvent] | solvent] | [μg/mL solvent] |
| α-Pinene | 8.2 | 0.9993 | 7.57 | 0.53 | 0.5-250 |
| Camphene | 9.2 | 0.9995 | 6.36 | 0.48 | 0.5-250 |
| β-Pinene | 10.6 | 0.9992 | 8.39 | 1.91 | 0.5-250 |
| β-Myrcene | 11.5 | 0.9996 | 5.45 | 0.96 | 0.5-250 |
| δ-3-Carene | 12.9 | 0.9999 | 1.46 | 0.77 | 0.5-250 |
| α-Terpinene | 13.4 | 0.9994 | 7.08 | 2.2 | 0.5-250 |
| p-Cymene | 13.8 | 0.9994 | 7.23 | 0.90 | 0.5-250 |
| d-Limonene | 14.0 | 0.9995 | 6.11 | 0.44 | 0.5-250 |
| Ocimene | 15.3 | 0.9996 | 5.98 | 3.77 | 0.5-250 |
| γ-Terpinene | 16.4 | 0.9996 | 5.30 | 0.99 | 0.5-250 |
| Terpinolene | 18.0 | 0.9995 | 6.25 | 0.31 | 0.5-250 |
| Linalool | 19.6 | 0.9997 | 4.65 | 2.67 | 0.5-250 |
| (−)-Isopulegol | 23.1 | 0.9996 | 5.32 | 3.30 | 0.5-250 |
| Geraniol | 30.8 | 0.9988 | 20.35 | 11.12 | 0.5-250 |
| β- | 38.9 | 0.9997 | 4.91 | 0.84 | 0.5-250 |
| Caryophyllene | | | | | |
| α-Humulene | 40.6 | 0.9996 | 5.33 | 0.94 | 0.5-250 |
| Nerolidol | 46.9 | 0.9957 | 22.70 | 13.19 | 0.5-250 |
| (−)-Guaiol | 48.7 | 0.9996 | 5.72 | 4.04 | 0.5-250 |
| (−)-α-Bisabolol | 54.1 | 0.9989 | 9.58 | 4.75 | 0.5-250 |

Here, RT relates to retention time in minutes, LOQ relates to limit of quantitation, and LOD relates to limit of detection.

### The LC-PDA-MS/MS Analysis of Cannabinoids

The molecular mass, elemental composition, and major molecular fragments of the unknown phytocannabinoids UK2.09, UK5.5, and UK7.45 were identified as CBTA, CBGMA, and THCA-C4, respectively. This was done by using LC-PDA-MS/MS analysis in negative mode. LC-PDA-MS/MS analysis was performed using the same mobile phase and column used for the HPLC-PDA cannabinoid quantification. In brief, samples were analyzed using an LC-MS/MS system, which consisted of a Dionex Ultimate 3000 RS HPLC coupled to a Q Exactive Plus hybrid FT mass spectrometer equipped with a heated electrospray ionization source (Thermo Fisher Scientific, USA). The HPLC system consisted of a quaternary pump, a thermostated autosampler, a thermostated column compartment, and a PDA detector. The HPLC separations were carried out using a Kinetex SB C18 column (2.1×150 mm, particle size 1.6 μm, Phenomenex). The mass spectrometer was operated in negative and positive ionization modes. The ion source parameters were as follows: spray voltage 3.5 kV, capillary temperature 300° C., sheath gas rate (arb) 40, and auxiliary gas rate (arb) 10. Mass spectra were acquired in the m/z 150-800 Da range at a resolving power of 70.000. The collision-induced fragmentations were acquired at 40 Normalized Collision Energy (NCE) values. The LC-MS system was controlled, and the data were analyzed using Xcalibur software (Thermo Fisher Scientific, USA).

### Chemometrics

A preprocessing transformation (PPT) was applied as a crucial first model-development step. Spectral PPTs are used to remove inappropriate information that the modeling techniques cannot handle correctly. Preprocessing is to linearize the variables' responses and remove extraneous sources of variance that are not of interest in the analysis. We applied several common PPTs to the raw data, including preprocessing smoothing operations such as Savitzky-Golay smoothing (first-order polynomial, 15/10 points per window or second-order polynomial, 15/10 points per window), first and second derivatives, standard normal variate (SNV), and multiplicative scatter correction (MSC), followed by normalization methods such as mean centering and/or autoscaling. In addition, removing data points below selected thresholds, using generalized least square-weighting (GLS-W) as a multivariate filtering technique was explored after smoothing and/or normalization operations had been carried out. After applying the aforementioned methods, we concluded that autoscaling followed by thresholding using GLS-W yielded the most accurate PLS-R and PLS-DA models for most compounds. However, the PLS-R mid- and low-range THCA sub-models and high-range CBDA sub-models required a smoothing preprocessing step before autoscaling.

In this connection, Savitzky Golay smoothing may be performed by conventional modules on a matrix of row vectors y. At each increment (column), a polynomial of order is fitted to the number of points widths surrounding the increment. An estimate for the function's value or derivative at the increment is calculated from the fit resulting in a smoothed function.

Standard Normal Variate (SNV) normalization method provides a weighted normalization (such that not all points contribute to the normalization equally). SNV utilizes the standard deviation of all the pooled variables for a given sample. The entire sample is then normalized by the value of the standard deviation, thus giving the sample a unit standard deviation (s=1). The technique utilizes determining mean and standard deviation.

The next step was to apply a multivariate statistical analysis using PLS-DA to classify the major medicinal cannabis cultivars available in Israel (i.e., high THC, high CBD, high CBG, and hybrid) and to classify the 15 different chemovars used in the present study, namely, 73-12, 523, 516, 512, 505, 45-22, 240, 236, 212, 159-3, 159-1, 146, 145-9, 145-13, and 141-3. PLS-DA was performed using 325 samples from 15 different cultivars, and their corresponding spectra were measured between 1000-2500 nm based on FT-NIR. PLS-DA enabled major class prediction by creating a Y-block of dependent variables for each item using a threshold line (estimated using Bayes' Theorem) above which the sample was considered related to the class. To test model generality and robustness and to avoid over-fitting, the PLS-DA model was cross-validated using the Venetian Blinds method, followed by an independent prediction test (i.e., n=237 for the calibration/validation group split ratio; 67%/33% of samples, respectively) and n=88 for the independent prediction group). The following parameters determined the performance of the cross-validity and predictability of the PLS-DA model: total accuracy, specificity, sensitivity, root mean standard error of calibration (RMSEC), root mean standard error of cross-validation (RMSECV), and root mean standard error of prediction (RMSEP). PLS-DA was performed using MATLAB and PLS Toolbox 8.9.

Based on their corresponding spectral signals, the PLS-R method was used to develop regression models of cannabinoids and terpenes. The PLS1 algorithm provided by the PLS Toolbox 8.9 software was used with the FT-NIR spectra in this study. The spectral and concentration data were first encoded in matrix form and then reduced to a few latent-variable (LV) factors. Therefore, the resulting spectral vectors were directly related to the cannabinoid and terpene concentrations. The number of LVs required to model the data was chosen based on optimal performance parameters for generating a predictive model, as described below. As for the PLS-DA model, the PLS-R models were cross-validated using the Venetian Blinds method, followed by an independent prediction test. Finally, validation errors were combined to obtain RMSEC and RMSECV values. To exclude outliers, cross-validation (CV) residuals, leverages, Q residuals, and Hotelling's T² were calculated. Samples that presented high leverages (>3× population mean), Hotelling's T² (T² reduced value >2), and residuals (Stdnt residuals ˜3/−3) were excluded from the model. The final models were built using the specific bands that exerted the greatest impact on the model, using the variable importance in projection (VIP) method. Practically, the VIP score is the ratio between the ability of a predictor to explain the variation orthogonal response variables and its covariance with the overall LVs. The typical cutoff for VIP influence is 1, the average of the squared scores.

An external validation dataset was used to assess the PLS-R models' predictive ability, utilizing a simple regression between FT-NIR predicted values and reference data. Residual predictive deviation (RPD) was calculated by the ratio of laboratory standard deviation to the RMSEP, and the ratio of performance to inter-quartile distance (RPIQ) statistics to evaluate the models' robustness. The best model was selected for each cannabinoid and terpene according to the highest R²_{cv}, R²ₚᵣₑ, RPIQ, and RPD values; the lowest RMSECV and RMSEP values, and the proximity of the ratio RMSECV/RMSEC to 1. Graduated ranking of the prediction models based on RPD is suggested by the conventional techniques. This includes ranking models into three main categories, with RPD≥2.5 and R²≥0.80 considered excellent, 2<RPD≤2.5 and R²≥0.70 considered good, 1.5<RPD≤2 and R²≥0.60 considered moderate, and RPD≤1.5 and R²<0.60 considered poor.

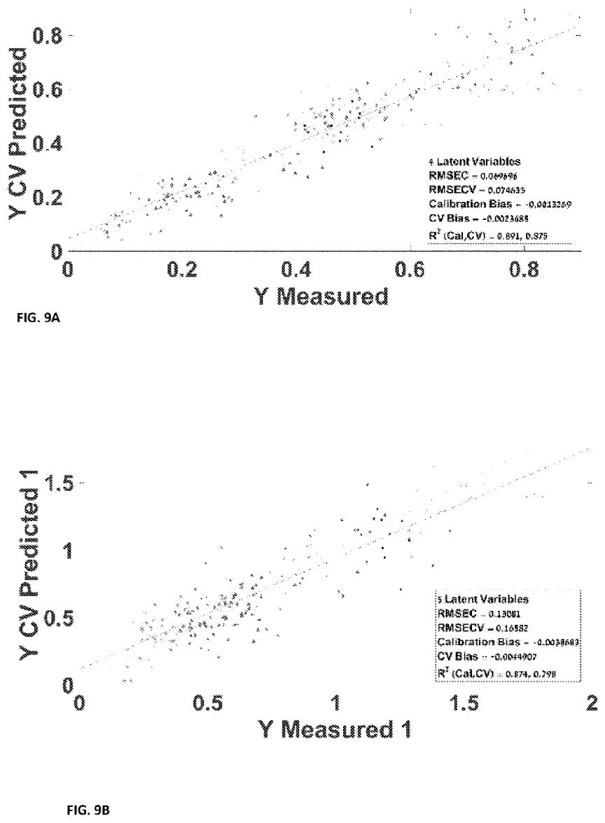

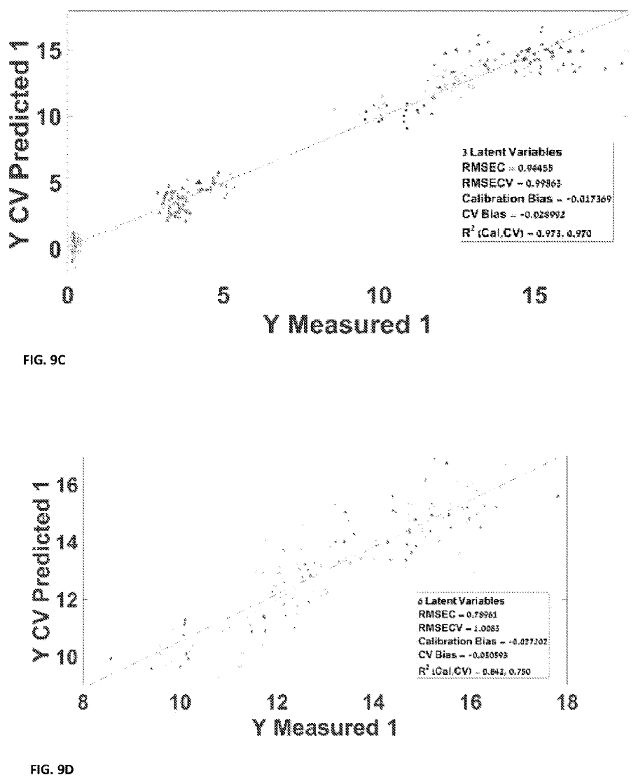

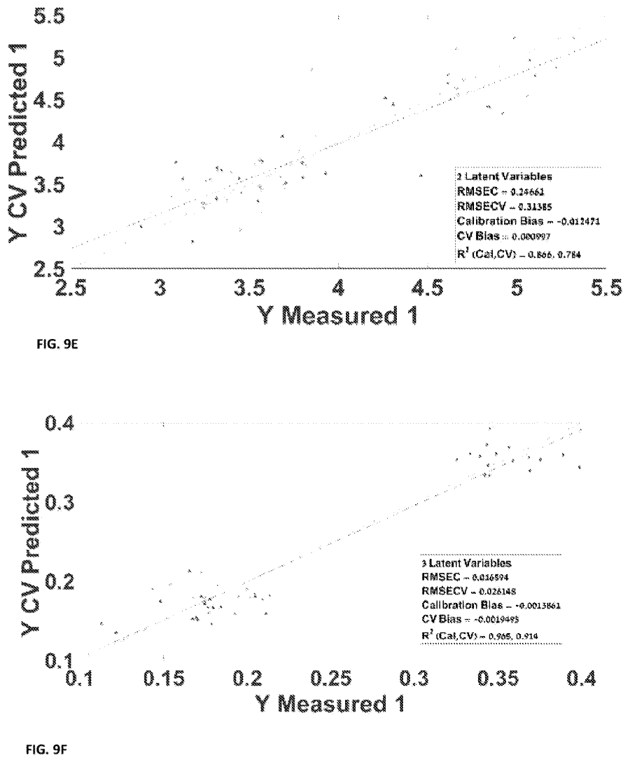

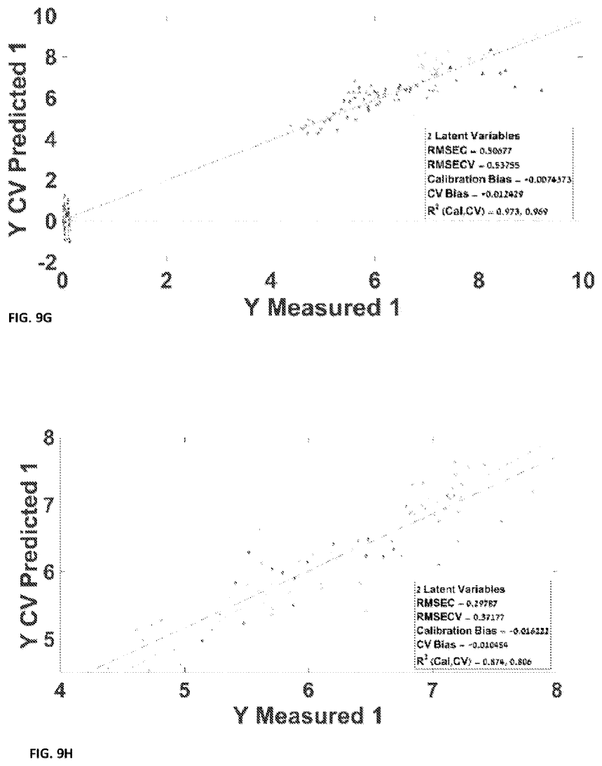

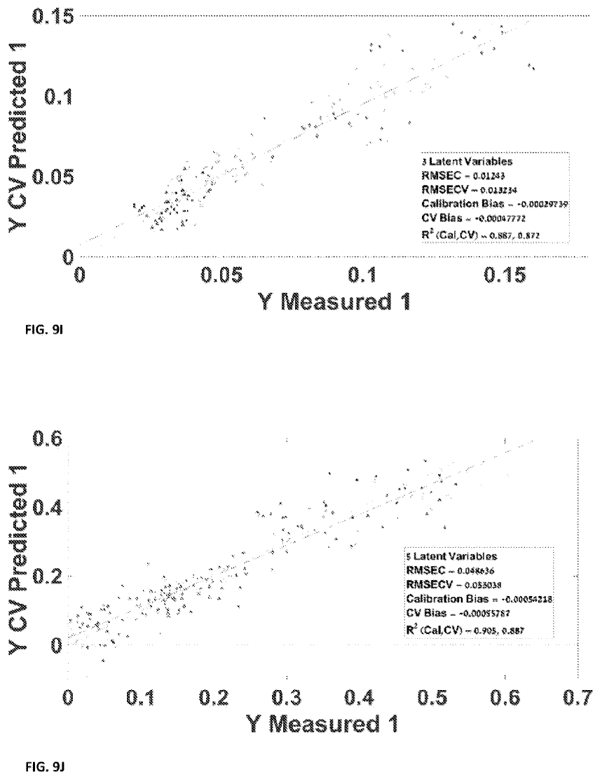

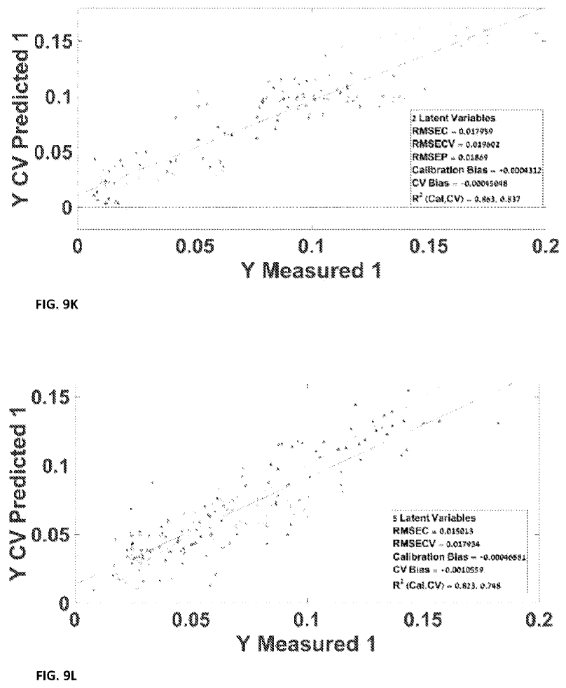

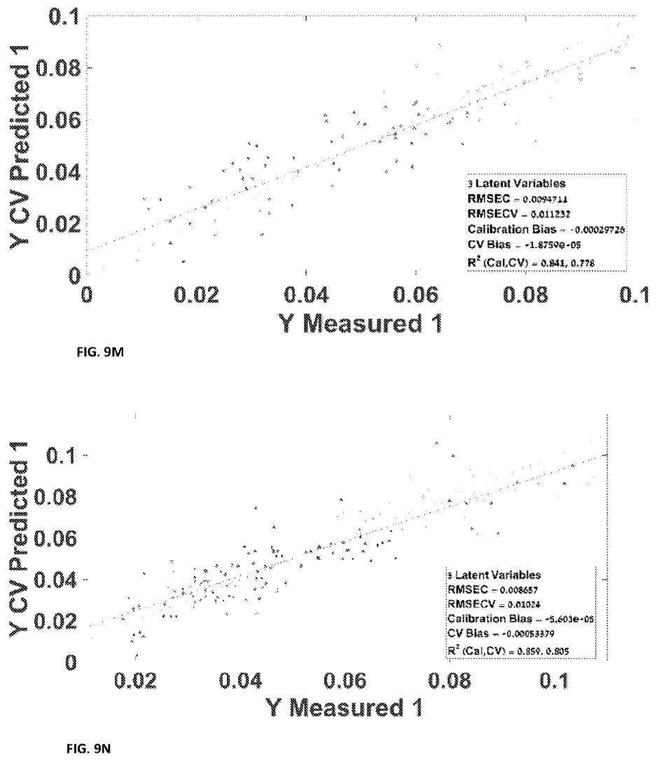

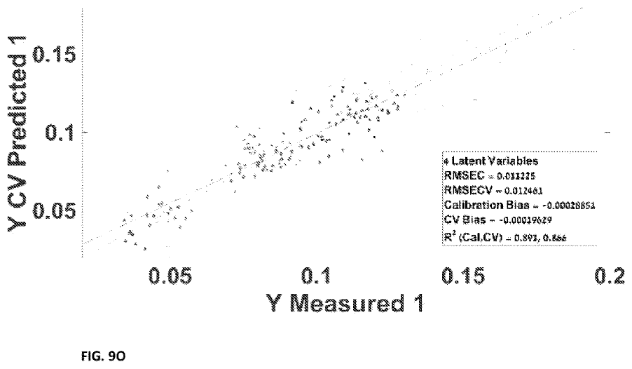

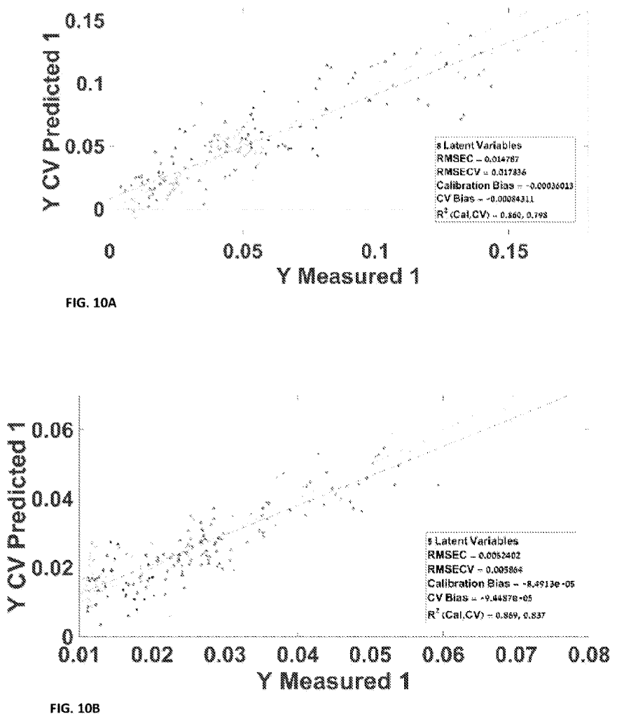

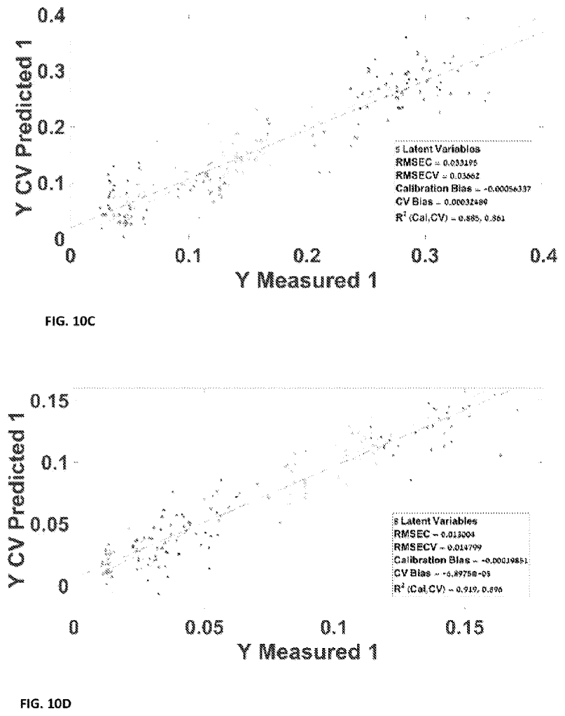

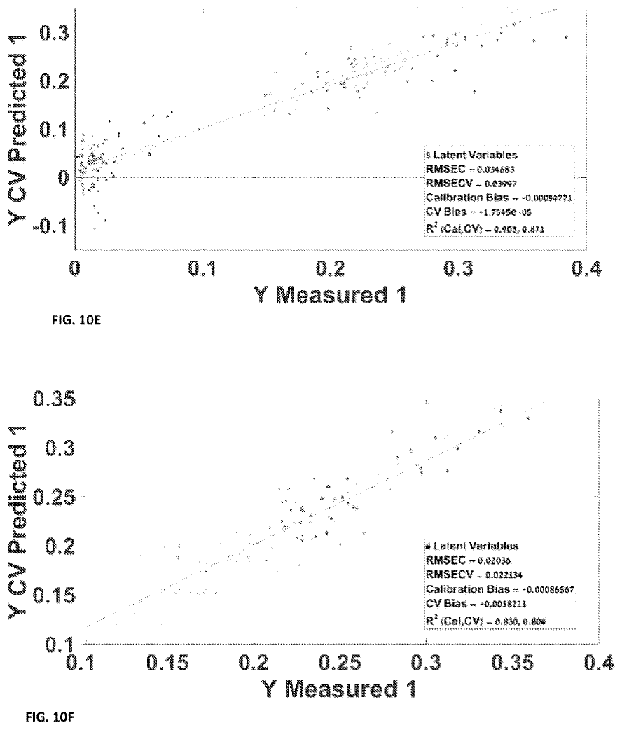

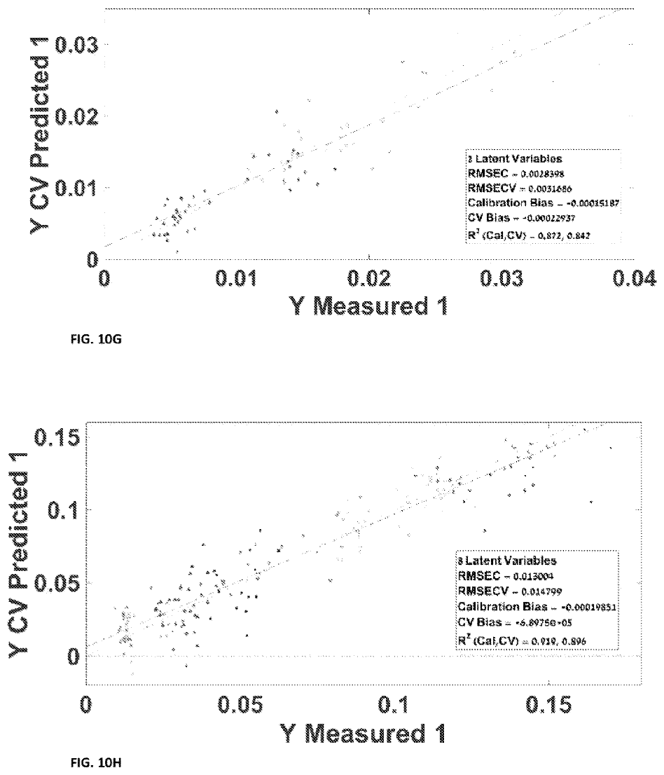

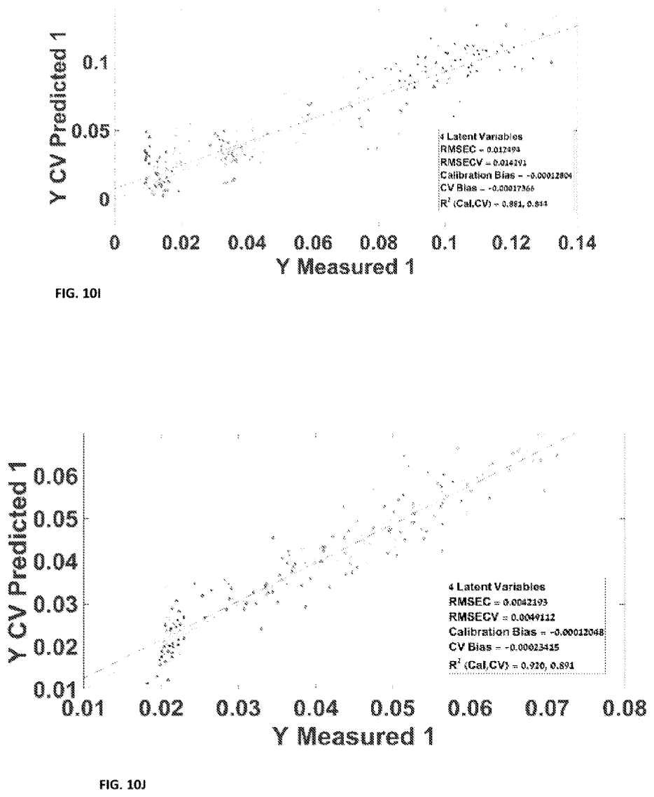

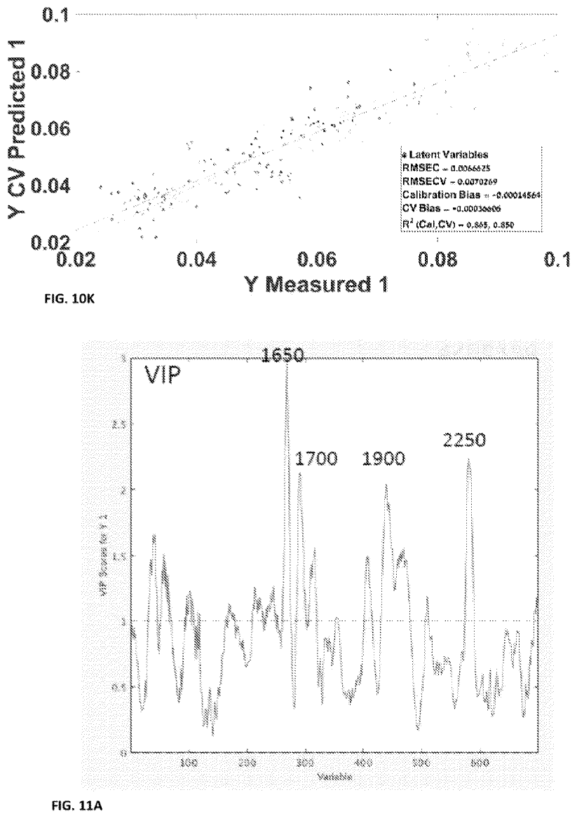

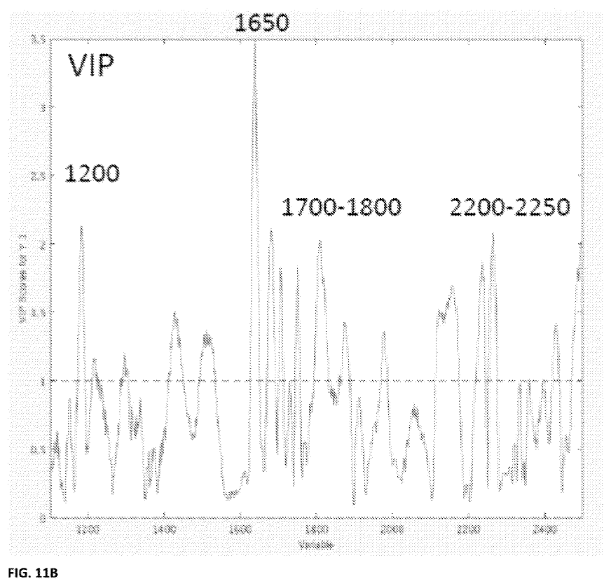

PLS-R models revealing substantial gaps in the correlation curves between measured and predicted concentrations were subdivided to cover only the concentration range for which inflorescence samples were available. For example, the PLS-R model for THCA was subdivided into a high range, mid-range, and low range, whereas the PLS-R models for CBDA and α-pinene were subdivided into high- and low-range models (FIGS. 9 and 10 and Tables 4 and 5). Subsequently, the subdivided models' performance was compared to the full-range models, and the optimal model was selected (FIGS. 9 and 10 and Tables 4 and 5).

### Results and Discussion

### Average Cannabinoid and Terpene Concentrations of Each Chemovar

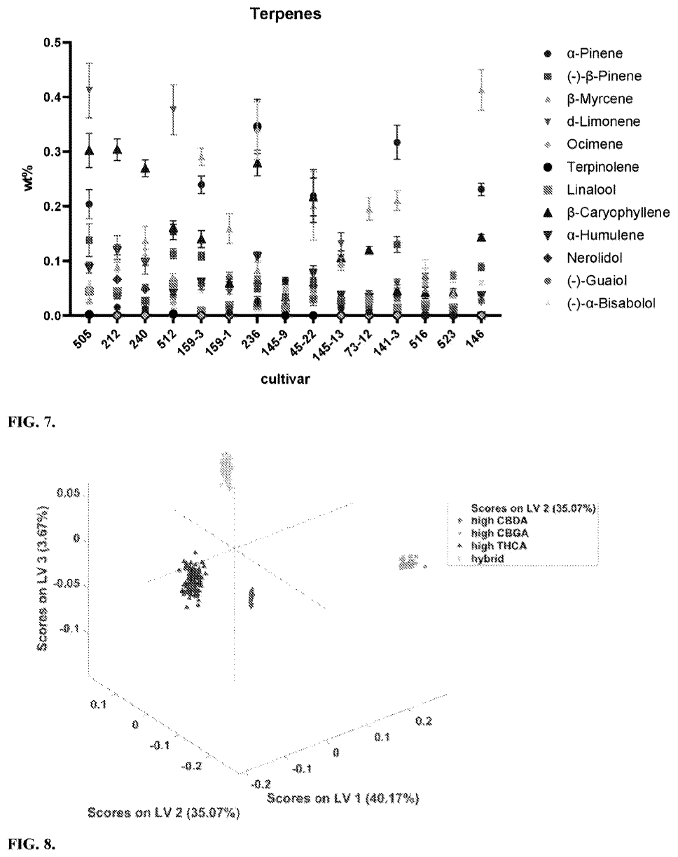

The average concentrations (+standard deviation) of cannabinoids and terpenes in each of the studied chemovars are presented in FIGS. 6, and 7. Altogether, 10 cannabinoids and 12 terpenes were identified and quantified in the cannabis inflorescence samples using HPLC-PDA and GC-MS, respectively (FIGS. 6 and 7). The first seven chemovars (505, 212, 240, 512, 159-3, 159-1, and 236) were characterized by a total THC to total CBD ratio>100, as well as low average minor cannabinoid concentrations (i.e., ≤1%). Consequently, the latter chemovars could be assigned to the high-THC class, according to the Cannabis Regulatory Unit of the Israeli Ministry of Health (FIG. 6). Only two high-THCA chemovars did not exhibit a statistical difference in all of their major cannabinoids (159-1 and 236). On the other hand, no common denominator could be identified regarding their terpene profiles. Each chemovar had a unique terpene profile (FIG. 6).

Except for chemovars 73-12 and 141-3, which had similar major cannabinoid concentrations, the majority of the hybrid chemovars differed significantly from one another in their cannabinoid contents, namely, in their levels of THCA, CBDA, CBGA, CBCA, THC, and CBD (FIG. 6). Moreover, chemovars 73-12 and 141-3 had a completely different terpene profiles (FIG. 7). Furthermore, the hybrid chemovars are characterized by a total THC to total CBD ratio of 0.8<ratio<1.25, which meets the definition of the hybrid classification according to the Israeli Cannabis Regulatory Unit. As for the high-THC chemovars, the terpene profile for each of the hybrid chemovars was unique, and no uniformity between the various terpenes could be discerned among the hybrid chemovars (FIG. 7).

The two high-CBG chemovars, 516 and 523, displayed identical cannabinoid profiles, with CBGA concentrations of one to three orders of magnitude greater (>15% by weight) than the concentrations of the remaining minor cannabinoids. Moreover, their total THC levels were below 0.3%. Hence the high-CBG chemovars can also be defined as hemp, according to the US-FDA cannabis cultivar definition. These chemovars also had similar terpene profiles, with the exception of β-myrcene.

In contrast, Chemovar 146 had a total THC to total CBD ratio<0.08 and a total CBD concentration>10%. Therefore, according to the Israeli Cannabis Regulatory Unit, it can be classified as a high-CBD cultivar. The average concentrations of the less prominent cannabinoids, including total THC, were below 1%, imparting the chemovar hemp status. Moreover, the terpene profile of the high-CBD chemovar differed significantly from the remaining chemovars, giving it a unique terpene profile (FIGS. 7 and 8).

### PLS-DA Classification by Major Class and by Chemovar

PLS-DA classification of the medicinal cannabis inflorescence samples into four major classes (i.e., high THC, high CBD, high CBG, and hybrid) was performed solely on the basis of the FT-NIR spectrum of each of the dried, homogenously ground cannabis inflorescence samples. That PLS-DA classification yielded an absolute class separation and perfect class prediction, using only three latent variables (FIG. 8). The calibration, cross-validation, and prediction groups had sensitivity and specificity values of 1. That is, no misclassification errors were observed. Sensitivity is related to the number of samples of a single chemovar that were correctly classified, whereas specificity is related to the number of samples that do not belong to a certain chemovar that were correctly classified. Specificity and sensitivity values approaching unity indicate a highly accurate classification model. The RMSEC, RMSECV, and RMSEP values for the four major classes ranged between 0.0187 and 0.0951. The RMSECV/RMSEC and RMSEP/RMSECV ratios were below 1.5, which is indicative of a low probability of model overfitting to the data. Overall, the PLS-DA model accurately classified all major cannabis classes.

TABLE 3
Cross-validation and prediction performance parameters of the PLS-DA chemovar classification model.

| | measured | | | | | | | | | | | | | | |
|---|---|---|---|---|---|---|---|---|---|---|---|---|---|---|---|
| Predicted | 141-3 | 145-13 | 145-9 | 146 | 159-1 | 159-3 | 212 | 236 | 240 | 45-22 | 505 | 512 | 516 | 523 | 73-12 |
| Major | Hy- | Hy- | Hy- | High | High | High | High | High | High | Hy- | High | High | High | High | Hy- |
| class | brid | brid | brid | CBDA | THCA | THCA | THCA | THCA | THCA | brid | THCA | THCA | CBGA | CBGA | brid |
| Sensitivity | 0.714 | 0.286 | 0.571 | 0.938 | 1 | 0.938 | 0.615 | 0.928 | 1 | 0.867 | 1 | 1 | 0.750 | 0.467 | 0.938 |
| (cross- | | | | | | | | | | | | | | | |
| validation) | | | | | | | | | | | | | | | |
| Specificity | 0.991 | 0.982 | 1 | 1 | 0.991 | 1 | 1 | 0.996 | 1 | 0.946 | 0.968 | 1 | 0.964 | 0.982 | 1 |
| (cross- | | | | | | | | | | | | | | | |
| validation) | | | | | | | | | | | | | | | |
| Accuracy | 0.975 | 0.941 | 0.987 | 0.996 | 0.991 | 0.996 | 0.979 | 0.991 | 1 | 0.940 | 0.970 | 1 | 0.950 | 0.950 | 0.996 |
| (cross- | | | | | | | | | | | | | | | |
| validation) | | | | | | | | | | | | | | | |
| Sensitivity | 0.33 | 0.80 | 0.67 | 1 | 1 | 1 | 1 | 1 | 1 | 1 | 1 | 1 | 0.74 | 0.60 | 0.75 |
| (prediction) | | | | | | | | | | | | | | | |
| Specificity | 1 | 0.952 | 1 | 1 | 1 | 1 | 1 | 1 | 1 | 0.976 | 0.987 | 1 | 0.976 | 0.988 | 1 |
| (prediction) | | | | | | | | | | | | | | | |
| Accuracy | 0.955 | 0.944 | 0.989 | 1 | 1 | 1 | 1 | 1 | 1 | 0.977 | 0.989 | 1 | 0.966 | 0.966 | 0.989 |
| (prediction) | | | | | | | | | | | | | | | |
| RMSEC¹ | 0.213 | 0.218 | 0.136 | 0.133 | 0.208 | 0.221 | 0.181 | 0.127 | 0.155 | 0.232 | 0.206 | 0.153 | 0.175 | 0.184 | 0.161 |
| RMSECV² | 0.213 | 0.219 | 0.137 | 0.146 | 0.208 | 0.221 | 0.184 | 0.131 | 0.160 | 0.232 | 0.207 | 0.160 | 0.177 | 0.184 | 0.166 |
| RMSEP³ | 0.223 | 0.215 | 0.146 | 0.130 | 0.219 | 0.198 | 0.212 | 0.127 | 0.174 | 0.220 | 0.228 | 0.159 | 0.159 | 0.162 | 0.159 |

¹RMSEC, standard error of calibration.
²RMSECV, standard error of cross-validation.
³RMSEP, standard error of prediction.

The chemovar PLS-DA classification model yielded poorer separation and class prediction than the major-class prediction model (FIGS. 9A to 9O and Tables 3 and 4). Unlike the major class classification, which resulted in complete separation (FIG. 8), many chemovars belonging to the same major class formed inseparable clusters, which hindered sufficient separation and, therefore, precise chemovar prediction (FIGS. 9A to 9O and Table 4). This implies that these chemovars' spectral signatures are similar due to their chemical or genetic similarity. Moreover, each cluster was comprised of chemovars belonging to the same major cannabis class, implying similarity in their chemical composition. Consequently, the false-positive classifications were associated solely with chemovars of the same class (FIGS. 9a to 9O and Tables 3 and 4). According to two-way ANOVA, the major cannabinoid compositions of each of the four clustered major cannabis groups were comparable (p>0.05), while the high-CBGA chemovars displayed similar terpene compositions (p>0.05). Previous studies have demonstrated that chemovars descending from the same cultivar might share a high degree of genetic resemblance and, consequently, similar secondary metabolite compositions, which could result in overlapping classes in the PLS-DA classification. Therefore, the strong similarities between certain chemovars of the same major class could be due to their genetic similarity manifested as metabolite composition. Thus, the present classification tool may provide a fast and practical tool for breeders as they select desirable chemovars for further assessment, saving precious time and other resources.

The highest average sensitivity, specificity, and accuracy for both cross-validation and prediction models were obtained for chemovars from the high-THCA class (Table 3), followed by chemovars from the high-CBDA and hybrid classes. In contrast, the high-CBGA chemovar classification model exhibited the lowest performance-parameter values (Table 3). Moreover, the chemovars with the greatest sensitivity, specificity, and accuracy values of 1 belonged to the high-THCA class (Table 3). These results suggest that among the four major classes chemovars from the high-THCA class are classified most accurately with respect to other classes. On the other hand, the high-CBGA chemovars may be relatively poorly classified due to their similar cannabinoid and terpene compositions. The successful classification of a chemovar depends not only on its cannabinoid composition but also on the combination of its terpene and cannabinoid profiles, as both compound classes profoundly affect the performance of the PLS-DA models presented here. For instance, although the chemovars 159-1 and 236 (assigned to the high-THCA class) and 73-12 and 141-3 (assigned to the hybrid class) did not display statistical differences in all of their major cannabinoids, other chemovars from the same major classes, namely 212 (high THCA) and 145-13/145-9 (hybrid), displayed lower classification parameter values, despite significant differences in their major-cannabinoid profiles (Table 3).

This apparent discrepancy can be resolved by analyzing the terpene compositions of the less-separable chemovars, which substantially affected the classification performance. This demonstrates that terpene and cannabinoid composition should preferably be considered for improved chemovar identification. The RMSEC, RMSECV, and RMSEP values for chemovar classification ranged between 0.127 and 0.232 (Table 3). That is one order of magnitude higher than the major class classification model (Table 3), indicating that the cultivar classification was less accurate. The lowest average RMSEs were obtained for high-THCA and high-CBGA chemovars, whereas the highest average RMSEs were obtained for the hybrid chemovars (Table 3). The RMSECV/RMSEC and RMSEP/RMSECV ratios were close to 1, pointing at a low probability of model overfitting to the data. Overall, the PLS-DA model for major-class prediction was more reliable than the chemovar classification.

Table 4 shows Cross-validation confusion table obtained by the PLS-DA classification of cannabis according to chemovars. Colored cells represent the four different chemovar clusters: red, blue, green, and yellow cells represent hybrid, high-THCA, high-CBGA, and high-CBDA clusters, respectively.

| | actual | | | | | | | | | | | | | | |
|---|---|---|---|---|---|---|---|---|---|---|---|---|---|---|---|
| Predicted | 141-3 | 145-13 | 145-9 | 146 | 159-1 | 159-3 | 212 | 236 | 240 | 45-22 | 505 | 512 | 516 | 523 | 73-12 |
| 141-3 | 10 | 1 | | | | | | | | 1 | | | | | |
| 145-13 | 3 | 4 | | | | | | | | 1 | | | | | |
| 145-9 | | | 4 | | | | | | | | | | | | |
| 146 | | | | 15 | | | | | | | | | | | |
| 159-1 | | | | | 14 | | 1 | 1 | | | | | | | |
| 159-3 | | | | | | 15 | | | | | | | | | |
| 212 | | | | | | | 8 | | | | | | | | |
| 236 | | | | | | 1 | | 13 | | | | | | | |
| 240 | | | | | | | | | 32 | | | | | | |
| 45-22 | 1 | 9 | | 1 | | | | | | 13 | | | | | 1 |
| 505 | | | 3 | | | 4 | 4 | | | | 20 | | | | |
| 512 | | | | | | | | | | | | 15 | | | |
| 516 | | | | | | | | | | | | | 12 | 8 | |
| 523 | | | | | | | | | | | | | 4 | 7 | |
| 73-12 | | | | | | | | | | | | | | | 15 |

### PLS-R Model for Cannabinoid Prediction

For each of the cannabinoids and terpenes, specific VIP bands were used for PLS-R model construction (Tables 5A, 5B, 6A, 6B). FIGS. 11A and 11B show VIP bands for THC and THCA respectively. As shown, relevant absorption wavelengths provide improved specificity for different cannabinoids and similarly for Terpenes, enabling improved operation of the machine learning module. The spectral regions of 1450-1880 and 2130-2350 nm were identified as crucial for predicting all cannabinoids and terpenes, while the region 1000-1210 nm was crucial for only a few compounds (Tables 5A, 5B, 6A, 6B). Many of the VIP bands that had a value >1 were found to correspond to chemical bonds found in the detected terpenes and cannabinoids, as shown in Tables 5 and 6. In model evaluation, the PLS-R model that met all of the performance parameter values was considered to have a high predictive capability: R²_{CV} and R²_{pred}>0.8, RPD>2.5 and RPIQ>3, and an RMSECV/RMSEC ratio<1.2. PLS-R models that met all of the performance parameters of the following range were considered suitable for initial screening purposes: R²_{CV}>0.7 and R²_{pred}<0.8, RPD>2 and RPIQ<3, and 1.2<RMSECV/RMSEC ratio<2. Except for the low-THCA model, all cannabinoid and terpene models had RMSECV/RMSEC ratios lower than 1.28, indicating that these preliminary models allow prediction with an error rate of less than 30% for all of the studied compounds (Tables 5A, 5B and 6A, 6B). Moreover, only three cannabinoid models had RPD values lower than 2 and RPIQ values lower than 3, indicating that the vast majority of the models were robust and provided accurate predictions (Tables 5A and 5B).

In terms of terpenes, only two models had RPD values lower than 2 and RPIQ values lower than 3 (Tables 6A and 6B). The PLS-R models of the following cannabinoids and terpenes were found to be highly predictive: THCA (full-range model), CBDA (full-range model), CBCA, THC, α-pinene (full-range model), β-pinene, β-myrcene, linalool, guaiol, bisabolol, and caryophyllene (FIGS. 5 and 6 and Tables 5A, 5B, 6A, 6B). The PLS-R models of the following cannabinoids and terpenes were considered suitable for initial screening purposes: THCA (high-, mid-, and low-range models), CBDA (high- and low-range models), CBGA (full-range and low-range models), CBG, CBD, CBTA, CBGMA, THCA-C4, α-pinene (high- and low-range models), D-limonene, and α-humulene (FIGS. 5 and 6 and Tables 5A, 5B, 6A, 6B). Taken together, the full-range PLS-R models were found to be superior to the subdivided models for all of the relevant compounds, in terms of the performance parameters (i.e., R²_{CV}, R²_{pred}, RPD, RPIQ, and RMSECV/RMSEC ratio).

Notwithstanding, three of these models, namely the THCA, CBDA, and CBGA full-range models, were over-fitted due to the high variance explained (R²>0.97) and low bias. Therefore, splitting these models into submodels was essential to reduce the over-fitting.

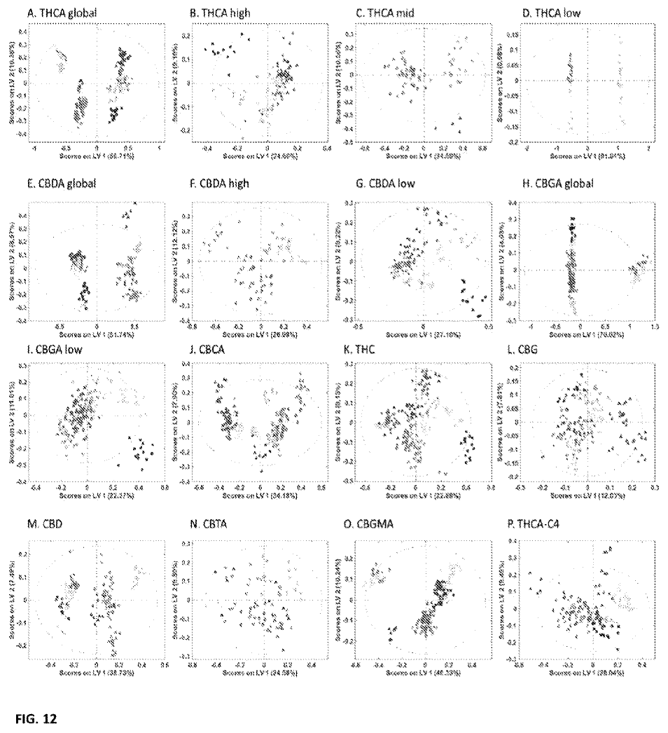

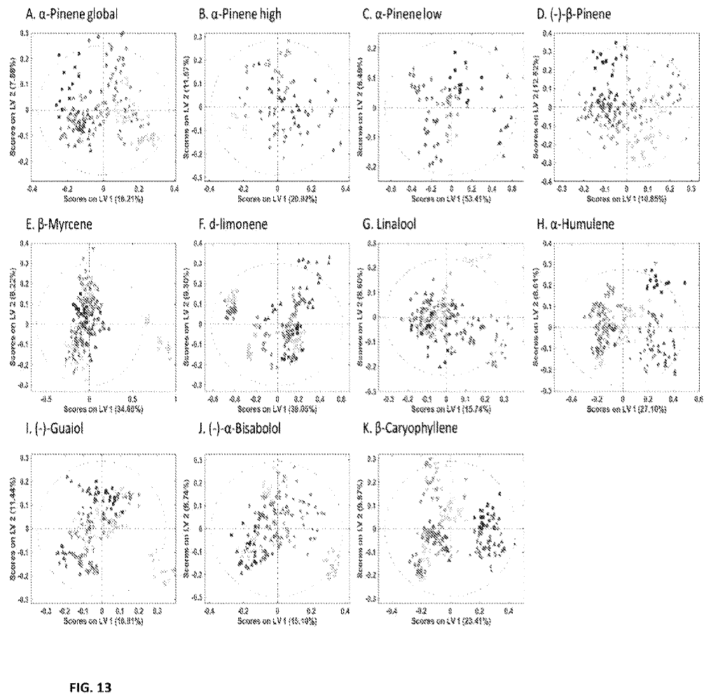

Examination of the PLS-R model score plots for the first two LVs is shown in FIGS. 12 and 13. The score plots reveal that spectral signature coupled to specific compound concentration enabled the classification of certain chemovars and/or major cannabis classes, such as in the case of the following models: all THCA models, CBDA full-range model, all CBGA models, CBD, CBGMA, β-myrcene, linalool, guaiol, and bisabolol (FIGS. 12 and 13). Some models allowed a full separation according to major class (e.g., THCA and CBDA full-range models), while others enabled the classification of certain chemovars (e.g., β-myrcene, linalool, guaiol, and bisabolol). These results support the hypothesis that a more comprehensive chemical composition characterization of cannabis inflorescence coupled with FT-NIR will improve future chemovar-classification models.

In conclusion, FT-NIR is a valuable tool for highly accurate quantitative and qualitative analysis of samples that contain organic compounds with specific functional groups (e.g., C\\H, C\\C, C\\O, C\\N, Ph, N\\H, S\\H, and O\\H), such as cannabinoids and terpenes. The enormous advantages of the NIRS compared to chromatographic techniques are the simple and fast sample preparation, short analysis time, and low costs associated with its use. FT-NIR is widely used in the food, medical, cosmetics, polymer, petrochemical, and pharmaceutical industries. The results of the PLS-R showed good prediction ability for 19 cannabinoids and terpenes. This study tested a large number of active compounds, and we were able to classify most chemovars with a high degree of accuracy. The use of FT-NIR for the prediction of cannabinoid and terpene concentrations and the classification of cannabis cultivars could transform the entire industry's quality control process. Specifically, it could reduce operational costs (profitability), reduce the price of the final medicinal cannabis product, and serve as a rapid selection tool for breeding programs.

TABLE 5A
Calibration, cross-validation, and prediction model parameters for cannabinoids.

| | n cal | | | | | | | RMSECV/ | | RPD | RPIQ |
|---|---|---|---|---|---|---|---|---|---|---|---|
| | n pre | | | | | | | RMSEC | | (SD/ | (Q3 − Q1)/ |
| Properties | cv % | R²_{adj}Cal | R²_{adj}Val | R²_{adj}Pred | LVs | RMSEC | RMSECV | ratio | RMSEP | RMSEP) | RMSEP |
| THCA | 237, 88, | 0.973 | 0.970 | 0.951 | 3 | 0.944 | 0.999 | 1.06 | 1.275 | 4.54 | 7.84 |
| full-model | 33% cv | | | | | | | | | | |
| THCA | 124, 51, | 0.842 | 0.750 | 0.771 | 6 | 0.789 | 1.008 | 1.28 | 1.117 | 1.87 | 2.71 |
| high-model | | | | | | | | | | | |
| (9-18% wt) | 33% cv | | | | | | | | | | |
| THCA | 66, 23, | 0.866 | 0.784 | 0.718 | 2 | 0.247 | 0.314 | 1.27 | 0.340 | 1.94 | 3.82 |
| mid-model | 20% cv | | | | | | | | | | |
| (3-6% wt) | | | | | | | | | | | |
| THCA | 47, 14 | 0.965 | 0.914 | 0.950 | 3 | 0.0166 | 0.0261 | 1.57 | 0.0215 | 4.05 | 7.95 |
| low model | 20% cv | | | | | | | | | | |
| (0.15-0.4% wt) | | | | | | | | | | | |
| CBDA global | 237, 88 | 0.973 | 0.969 | 0.968 | 2 | 0.507 | 0.537 | 1.06 | 0.531 | 5.78 | 10.7 |
| | 33% cv | | | | | | | | | | |
| CBDA | 75, 28 | 0.874 | 0.806 | 0.839 | 2 | 0.298 | 0.372 | 1.25 | 0.344 | 3.95 | 2.44 |
| high model | 20% cv | | | | | | | | | | |
| (4-8% wt) | | | | | | | | | | | |
| CBDA | 155, 60, | 0.887 | 0.872 | 0.772 | 3 | 0.012 | 0.013 | 1.08 | 0.018 | 2.06 | 3.44 |
| low model | 33% cv | | | | | | | | | | |
| (0.02-0.15% wt) | | | | | | | | | | | |
| CBGA global | 237, 88, | 0.993 | 0.992 | 0.990 | 2 | 0.405 | 0.434 | 1.07 | 0.435 | 10.87 | 1.70 |
| | 33% cv | | | | | | | | | | |
| CBGA | 204, 79, | 0.874 | 0.798 | 0.731 | 5 | 0.131 | 0.166 | 1.27 | 0.182 | 2.03 | 2.51 |
| low | 33% cv | | | | | | | | | | |
| (0.2-2% wt) | | | | | | | | | | | |
| CBCA | 237, 88 | 0.891 | 0.875 | 0.875 | 4 | 0.070 | 0.075 | 1.07 | 0.075 | 2.81 | 4.41 |
| (0.05-1% wt) | 33% cv | | | | | | | | | | |
| THC | 217, 81, | 0.905 | 0.887 | 0.856 | 5 | 0.049 | 0.053 | 1.08 | 0.060 | 2.59 | 3.61 |
| (0.01-0.6% wt) | 33% cv | | | | | | | | | | |
| CBG | 186, 74, | 0.823 | 0.748 | 0.719 | 5 | 0.015 | 0.018 | 1.2 | 0.019 | 1.89 | 2.84 |
| (0.02-0.15% wt) | 33% cv | | | | | | | | | | |
| CBD | 132, 43, | 0.863 | 0.837 | 0.829 | 2 | 0.018 | 0.02 | 1.11 | 0.019 | 2.58 | 4.05 |
| (0.01-0.2% wt) | 33% cv | | | | | | | | | | |
| UK2.09 | 91, 27, | 0.841 | 0.778 | 0.653 | 3 | 0.0095 | 0.011 | 1.16 | 0.012 | 2 | 3.08 |
| (CBTA) | 20% cv | | | | | | | | | | |
| (0.01-0.1% wt) | | | | | | | | | | | |
| UK5.5 | 205, 78, | 0.891 | 0.866 | 0.809 | 4 | 0.011 | 0.012 | 1.12 | 0.014 | 2.5 | 3.09 |
| (CBGMA) | 33% cv | | | | | | | | | | |
| (0.02-0.2% wt) | | | | | | | | | | | |
| UK7.45 | 157, 56, | 0.859 | 0.805 | 0.625 | 5 | 0.0086 | 0.010 | 1.16 | 0.011 | 2.09 | 3.45 |
| (THCA-C4) | 33% cv | | | | | | | | | | |
| (0.02-1.25% wt) | | | | | | | | | | | |

TABLE 5B
Calibration, cross-validation, and prediction model parameters for cannabinoids.

| | VIP bands (nm) | | Outlier |
|---|---|---|---|
| Properties | (Bonds associated with VIP band) | VIP | number |
| THCA full-model | 1187 (CH3 CH2 CH), 1450 (CH ROH), 1640 (ArCH CH3), | 982 | 2 |
| | 1680 (CH3 CH2 CH), 1710 (CH3 CH2 CH), 1750 (CH2 CH), | | |
| | 1805, 2121, 2250-2270 (CH3), 2430 (CH) | | |
| THCA high-model | 1000, 1845-1880 (RCOOH), 2135, 2210 (CH3) | 534 | 7 |
| (9-18% wt) | | | |
| THCA mid-model | 1000, 1630 (ArCH CH3), 1925 (POH RCOOH), 2300 | 1012 | 3 |
| (3-6% wt) | (CH3 CH2 CH), 2500 | | |
| THCA low model | 1450 (CH ROH), 1640 (ArCH CH3), 1700 (CH3 CH2 CH), 2295 | 1012 | 0 |
| (0.15-0.4% wt) | (CH3 CH2 CH), 2470 | | |
| CBDA global | 1150 (CH3), 1520, 1640 (ArCH CH3), 1680 (CH3 CH2 CH), | 827 | 0 |
| | 1710 (CH3 CH2 CH), 1750 (CH2 CH), 2260 (CH3 CH2) | | |
| CBDA high model | 1960 (RCOOR), 2300 (CH3 CH2 CH) | 856 | 7 |
| (4-8% wt) | | | |
| CBDA low model | 1140 (CH3), 1465 (CH ROH), 1675 (CH3 CH2), 1970 (RCOOR), | 638 | 0 |
| (0.02-0.15% wt) | 2135, 2235 (CH3) | | |
| CBGA global | 1450 (CH ROH), 1635 (ArCH CH3), 1670 (CH3 CH2), 1715 | 1317 | 0 |
| | (CH3 CH2 CH), 2115, 2350 (CH3 CH2 CH), 2440, 2465 | | |
| CBGA low | 1215 (CH2 CH), 1640 (ArCH CH3), 1735 (CH2 CH), 2315, 2350 | 510 | 4 |
| (0.2-2% wt) | | | |
| CBCA | 1450 (CH ROH), 1505-1520, 1875 (RCOOH), 2255 (CH3 CH2), | 799 | 3 |
| (0.05-1% wt) | 2333 (CH3 CH2 CH), 2430 (CH) | | |
| THC | 1180 (CH3 CH2 CH), 1635 (ArCH CH3), 1680, 2135, 2265 | 763 | 3 |
| (0.01-0.6% wt) | | | |
| CBG | 1630 (ArCH CH3), 1930, 2035, 2235, 2500 | 408 | 3 |
| (0.02-0.15% wt) | | | |
| CBD | 1150, 1520, 1640 (ArCH CH3), 1810, 1870 (RCOOR), | 657 | 1 |
| (0.01-0.2% wt) | 2260 (CH3 CH2) | | |
| UK2.09 (CBTA) | 1810, 1985, 2305 (CH3 CH2 CH) | 735 | 1 |
| (0.01-0.1% wt) | | | |
| UK5.5 (CBGMA) | 1185 (CH3 CH2 CH), 1450 (CH ROH), 1675 (CH3 CH2), | 227 | 2 |
| (0.02-0.2% wt) | 1715 (CH3 CH2 CH), 2120, 2240 (CH3), 2250 (CH3 CH2), 2465 | | |
| UK7.45 | 1185, 1640 (ArCH CH3), 1705 (CH3 CH2 CH), 1750 (CH2 CH), | 227 | 5 |
| (THCA-C4) | 1970 (RCOOR), 2235 (CH3) | | |
| (0.02-1.25% wt) | | | |

N cal, n pred and cv %, sample size of the calibration, prediction dataset, respectively and percent confidence of variation; R²_{cal}, coefficient of determination for calibration; R²_{cv}, coefficient of determination for validation group; R²ₚᵣₑ, coefficient of determination for prediction group; LVs, number of latent variables; RMSEC, root mean square error of calibration; RMSECV, root mean square error of cross validation; RMSEP, root mean square error of prediction; RPD, residual predictive deviation, SD_{pred}/RMSEP ratio; RPIQ, ratio of performance to inter-quartile distance, (Q3 − Q1)/RMSEP.

TABLE 6A
Calibration, cross-validation, and prediction model parameters for terpenes.

| | n cal | | | | | | | RMSECV/ | | RPD | RPIQ |
|---|---|---|---|---|---|---|---|---|---|---|---|
| | n pre | | | | | | | RMSEC | | (SD/ | (Q3 − Q1)/ |
| Properties | cv % | R²_{adj}Cal | R²_{adj}Val | R²_{adj}Pred | LVs | RMSEC | RMSECV | ratio | RMSEP | RMSEP) | RMSEP |
| α-Pinene | 186, 71, | 0.903 | 0.871 | 0.866 | 5 | 0.035 | 0.040 | 1.14 | 0.041 | 2.73 | 5.12 |
| full-model | 33% cv | | | | | | | | | | |
| (0.003-0.4% wt) | | | | | | | | | | | |
| α-Pinene | 84, 32, | 0.872 | 0.842 | 0.813 | 2 | 0.0028 | 0.0032 | 1.14 | 0.0037 | 2.16 | 3.24 |
| low model | 33% cv | | | | | | | | | | |
| (0.003-0.04% wt) | | | | | | | | | | | |
| α-Pinene | 95, 33, | 0.830 | 0.804 | 0.700 | 4 | 0.0204 | 0.0221 | 1.08 | 0.0282 | 1.78 | 2.29 |
| high model | 33% cv | | | | | | | | | | |
| (0.13-0.4% wt) | | | | | | | | | | | |
| (−)-β-Pinene | 186, 71, | 0.919 | 0.896 | 0.880 | 8 | 0.013 | 0.015 | 1.15 | 0.017 | 2.70 | 5 |
| (0.01-0.18% wt) | 33% cv | | | | | | | | | | |
| β-Myrcene | 218, 80, | 0.903 | 0.895 | 0.887 | 5 | 0.0346 | 0.0360 | 1.04 | 0.0368 | 3 | 4.05 |
| (0.02-0.45% wt) | 33% cv | | | | | | | | | | |
| D-limonene | 183, 65, | 0.860 | 0.798 | 0.745 | 8 | 0.015 | 0.018 | 1.20 | 0.020 | 2 | 2.4 |
| (0.01-0.18% wt) | 33% cv | | | | | | | | | | |
| Linalool | 199, 74, | 0.869 | 0.837 | 0.843 | 5 | 0.0052 | 0.0059 | 1.13 | 0.0056 | 2.5 | 3.39 |
| (0.01-0.07% wt) | 33% cv | | | | | | | | | | |
| α-Humulene | 218, 80, | 0.881 | 0.844 | 0.845 | 4 | 0.0125 | 0.0143 | 1.14 | 0.0155 | 2.32 | 4.52 |
| (0.01-0.14% wt) | 33% cv | | | | | | | | | | |
| (−)-Guaiol | 178, 62, | 0.865 | 0.850 | 0.868 | 4 | 0.0067 | 0.0070 | 1.04 | 0.0066 | 2.73 | 4.24 |
| (0.02-0.1% wt) | 33% cv | | | | | | | | | | |
| (−)-α-Bisabolol | 164, 57, | 0.920 | 0.891 | 0.874 | 4 | 0.0042 | 0.0049 | 1.17 | 0.0050 | 3 | 5.8 |
| (0.01-0.08% wt) | 33% cv | | | | | | | | | | |
| β-Caryophyllene | 218, 80, | 0.885 | 0.861 | 0.850 | 5 | 0.0332 | 0.0366 | 1.10 | 0.0416 | 2.33 | 4.67 |
| (0.02-0.35% wt) | 33% cv | | | | | | | | | | |

TABLE 6B
Calibration, cross-validation, and prediction model parameters for terpenes.

| | | VIP | Outlier |
|---|---|---|---|
| Properties | VIP bands (nm) (bonds associated with VIP band) | number | number |
| α-Pinene full-model | 1630 (ArCH), 1675 (CH3 CH2 CH), 2165 (CH3), 2235 | 526 | 1 |
| (0.003-0.4% wt) | (CH3), 2330 (CH3 CH2 CH), 2365 (CH3 CH2 CH) | | |
| α-Pinene low model | 1630 (ArCH), 1925, 2065, 2195 (CC) | 118 | 4 |
| (0.003-0.04% wt) | | | |
| α-Pinene high model | 1630 (ArCH), 1635 (ArCH), 1710 (CH3 CH2 CH), 1735 | 208 | 4 |
| (0.13-0.4% wt) | (CH2 CH), 1750 (CH2 CH), 1890, 2200 (CH3 CC), 2255 | | |
| | (CH3 CH2), 2315 (CH3 CH2 CH), 2450, 2490 | | |
| (−)-β-Pinene | 1630 (CH3), 1640 (CH3), 1675 (CH3 CH2), 2135, 2160 (CC), | 360 | 1 |
| (0.01-0.18% wt) | 2235 (CH3), 2325 (CH3 CH2 CH), 2365 (CH3 CH2 CH) | | |
| β-Myrcene | 1450 (CH), 1630 (CH3), 1950, 2135 | 1034 | 0 |
| (0.02-0.45% wt) | | | |
| D-limonene | 1450 (CH), 1640 (ArCH CH3), 1675 (CH3 CH2), 2090, | 499 | 4 |
| (0.01-0.18% wt) | 2110, 2240 (CH3), 2465 | | |
| Linalool | 1640 (CH3), 2135, 2210, 2240 (CH3), 2255 (CH3), 2315 | 691 | 2 |
| (0.01-0.07% wt) | (CH3 CH2 CH), 2330 (CH3 CH2 CH) | | |
| α-Humulene | 1205 (CH2 CH), 1715 (CH3 CH2 CH), 2315 (CH3 CH2 CH) | 868 | 2 |
| (0.01-0.14% wt) | | | |
| (−)-Guaiol | 1140 (CH3), 1640 (CH3), 1835, 1950, 1975 | 603 | 1 |
| (0.02-0.1% wt) | | | |
| (−)-α-Bisabolol | 1635 (CH3), 1810, 1965, 2135, 2235 (CH3) | 842 | 1 |
| (0.01-0.08% wt) | | | |
| β-Caryophyllene | 1715 (CH3 CH2 CH), 1730 (CH2 CH), 2425 | 798 | 1 |
| (0.02-0.35% wt) | | | |

N cal, n pred and cv %, sample size of the calibration, prediction dataset, respectively and percent confidence of variation; R²_{cal}, coefficient of determination for calibration; R²_{cv}, coefficient of determination for validation group; R²ₚᵣₑ, coefficient of determination for prediction group; LVs, number of latent variables; RMSEC, root mean square error of calibration; RMSECV, root mean square error of cross validation; RMSEP, root mean square error of prediction; RPD, residual predictive deviation, SD_{pred}/RMSEP ratio; RPIQ, ratio of performance to interquartile distance, (Q3 − Q1)/RMSEP.

Thus, the present disclosure provides an accurate, fast, relatively cost-effective, and simple technique and respective system and method for classifying cannabis inflorescence to determine cannabinoid and terpene quantitative prediction models using NIR spectroscopy of the inflorescence. The present technique utilizes selected machine learning models, including but not limited to PLS-DA and R classification techniques. The present technique enables determining major class assignments for different cannabis cultivars and the concentrations of 10 cannabinoids and 9 terpenes in dried cannabis inflorescences. The results obtained and exemplified herein confirm that the present technique utilizes information in the FT-NIR spectra for determining chemical and botanic classification prediction. It should be noted that the examples in the present disclosure are based on a selected set of chemovars available to the inventors. Including additional chemovars in the dataset could improve machine learning prediction and predictability of present technique models.

It should be noted that the various features described in the various embodiments can be combined according to all possible technical combinations. It should also be understood that the present invention is not limited in its application to the details set forth in the description contained herein or illustrated in the drawings. The invention is capable of other embodiments and being practiced and carried out in various ways. Hence, it is to be understood that the phraseology and terminology employed herein are for the purpose of description and should not be regarded as limiting. As such, those skilled in the art will appreciate that the conception upon which this disclosure is based can readily be utilized as a basis for designing other structures, methods, and systems for carrying out the several purposes of the presently disclosed subject matter.

Those skilled in the art will readily appreciate that various modifications and changes can be applied to the embodiments of the invention as hereinbefore described without departing from its scope, defined in and by the appended claims.

1. A method for use in the classification of cannabis inflorescence, the method comprising:
(a) grinding said cannabis inflorescence;
(b) determining a spectrogram of ground cannabis inflorescence; and
(c) providing data indicative of said spectrogram to trained machine learning system, pretrained on classification of material composition of cannabis inflorescence, to thereby obtain output data indicative of at least one of composition of selected cannabinoids and terpenes in said cannabis inflorescence, and varieties of said cannabis inflorescence.

2. The method of claim 1, wherein said grinding said cannabis inflorescence comprises grinding said cannabis inflorescence after freezing in liquid nitrogen.

3. The method of claim 1, wherein grinding said cannabis inflorescence comprises grinding to a predetermine powder size in the range of 1-10 micrometer.

4. The method of claim 1, wherein said determining a spectrogram of ground cannabis inflorescence comprises obtaining a Fourier Transform Infrared spectroscopic absorption data of said ground cannabis inflorescence.

5. The method of claim 1, wherein said determining a spectrogram of ground cannabis inflorescence comprises using a monochromator spectrometer.

6. The method of claim 1, wherein said spectrogram comprises wavelength range between 1000 nm and 2500 nm.

7. The method of claim 1, further comprises preprocessing of said spectrogram, said processing comprises at least one of signal amplification and thresholding of the spectrogram data.

8. The method of claim 7, wherein said preprocessing further comprises applying smoothing operation on at least one of said spectrogram, first derivative and second derivative thereof.

9. The method of claim 1, wherein said trained machine learning system is trained on a labeled data set comprising a plurality of cannabis inflorescence of a plurality of cannabis cultivar/varieties labeled by respective chemovar of said plurality of cannabis inflorescence.

10. The method of claim 9, wherein said respective chemovar is determined by at least one mass spectrometry and chromatography measurement of said plurality of cannabis inflorescence.

11. The method of claim 9, wherein said trained machine learning system comprises a plurality of processing routes, each processing route being directed for quantifying a selected one of cannabinoids and terpenes in said cannabis inflorescence; and wherein said preprocessing comprises generating a plurality of cropped copies of said data indicative of said spectrogram, wherein each of said cropped copies is cropped around one or more characteristic wavelength ranges indicative of absorption of a respective one of said selected cannabinoids and terpenes in said cannabis inflorescence.

12. (canceled)

13. A system for classification of cannabis inflorescence, comprising comprising:
at least one processor; and
a memory unit, associated with and one or more input/output connection;
wherein said at least one processor is configured and operable for receiving input data indicative of one or more spectrograms taken from one or more cannabis inflorescence samples, and processing said input data to determine quantitative data on one or more cannabinoid and terpene composition of said one or more cannabis inflorescence; wherein said processing comprises utilizing at least one pre-trained machine learning module pretrained on the classification of a material composition of cannabis inflorescence.

14. The system of claim 13, wherein said processing further comprises preprocessing of input spectrogram, said preprocessing comprises at least one of signal amplification and thresholding of said one or more spectrograms.

15. The system of claim 14, wherein said preprocessing further comprises applying smoothing operation on said one or more spectrograms, first derivative and second derivative thereof.

16. The system of claim 13, wherein said at least one pre-trained machine learning module comprises a plurality of processing routes, each processing route being directed for quantifying a selected one of cannabinoids and terpenes in said cannabis inflorescence.

17. The system of claim 16, wherein said at least one processor is configured and operable for preprocessing said one or more spectrograms and for generating a plurality of cropped copies of said one or more spectrograms, wherein each of said cropped copies is cropped around one or more characteristic wavelength ranges indicative of absorption of a respective one of said selected cannabinoids and terpenes in said cannabis inflorescence.

18. (canceled)

19. The system of claim 13, further comprising an infrared spectrometer unit connectable to said at least one processor via one or more communication lines; said infrared spectrometer unit comprises a sample mount for holding a sample and is configured to selective measure sample absorption in a selected wavelength range within infrared spectrum thereby generating spectrogram data indicative of one or more spectrograms taken from one or more cannabis inflorescence samples and transmitting said spectrogram data to said at least one processor.

20. The system of claim 19, wherein said infrared spectrometer unit is a Fourier Transform Infrared spectrometer unit.

21. A computer implemented method for use in classification of cannabis inflorescence, the computer implemented method comprising:
(a) receiving input data indicative of one or more infrared spectrograms of cannabis inflorescence;
(b) processing said input data to determine at least one of composition of selected cannabinoids and terpenes in said cannabis inflorescence, and cultivar of said cannabis inflorescence; and
(c) generating output data indicative of said at least one of composition of selected cannabinoids and terpenes in said cannabis inflorescence, and varieties of said cannabis inflorescence;
wherein, said processing comprises operating at least one machine learning module, pretrained for classification of material composition of cannabis inflorescence, to determine quantitative data on selected number of cannabinoids and terpenes in said cannabis inflorescence.

22. The computer implemented method of claim 21, wherein said at least one machine learning module comprises a plurality of processing routes, each processing route being directed for quantifying a selected one of cannabinoids and terpenes in said cannabis inflorescence.

23. (canceled)

24. (canceled)

25. (canceled)
